(19) United States
(12) Patent Application Publication
(10) Pub. No.: US 2022/0289756 A1
(43) Pub. Date: Sep. 15, 2022

(54) COMPOUNDS AND COMPOSITIONS AS INHIBITORS OF PROTEIN KINASES

(71) Applicants: WAI KIT PANG, BELMONT, MA (US); HEEJIN KIM, BELMONT, MA (US)

(72) Inventors: WAI KIT PANG, BELMONT, MA (US); HEEJIN KIM, BELMONT, MA (US)

(21) Appl. No.: 17/692,153

(22) Filed: Mar. 10, 2022

Related U.S. Application Data
(60) Provisional application No. 63/159,969, filed on Mar. 11, 2021.

(51) Int. Cl. C07D 487/04; C07D 277/40; C07D 413/12; C07D 471/04; C07D 417/12; C07D 233/88

(52) U.S. Cl. CPC C07D 487/04; C07D 277/40; C07D 413/12; C07D 471/04; C07D 417/12; C07D 233/88

(57) ABSTRACT

The invention provides small molecule heteroaromatic compounds that are ATP-competitive tyrosine kinase inhibitors displaying a significant inhibitory potency towards resistant T315I ABL mutants. The compounds of the invention find a useful application in the treatment of BCR-ABL inhibitor resistant diseases, such as imatinib resistant chronic myelogenous leukemia.

### FIELD OF THE INVENTION

The present invention relates to heteroaromatic compounds and salts thereof, to methods of using such compounds in treating protein kinase-associated disorders such as immunologic and oncologic disorders, and to pharmaceutical compositions containing such compounds.

### BACKGROUND

15% of all adult leukemia are chronic myelogenous leukemia (CML), which is a hematological disorder that is characterized by the malignant expansion of the myeloid lineage. More than 90% of CML patients have a change in their chromosome pairs 9 and 22. Part of chromosome-9 at the abelson (ABL) location has disconnected and fused together at the breakpoint cluster region (BCR) location of chromosome-22. This translocation and fusion of this pair of genetic materials (BCR-ABL) resulting to a truncated chromosome 22q, that is known as the “Philadelphia chromosome”. This Bcr-Abl gene is constitutively active protein tyrosine kinase (PTK) and interacts with multiple cellular signaling pathways which in turn results in transformation and deregulated proliferation of cells [Lugo T. G. et al. Science, 1990, 247, 1079-1082]. Hence, this is a primary driver for the cause of CML. In addition to this, approximately 10% of acute lymphoblastic lymphoma (ALL) patients have identified to have such Philadelphia chromosome.

At the turn of the millennium, a breakthrough in science and medicine for the treatment for leukemia patients after the discovery of the first generation tyrosine kinase inhibitor (TKI), imatinib (Gleevec®), which dramatically improve the 5-year overall survival rate of patients with CML from ˜30% to >90%.

With the remarkable safety and efficacy of imatinib, patients can expect to live a normal life span. However, long term treatment of imatinib invariably led to the development of drug resistance due to point mutations of Bcr-Abl, which render imatinib to become ineffective in controlling the patient's disease. More than 90 different types of point mutations have since been identified. The following 7 mutations, G250, Y253, E255, T315, M351, F359, and H396 are responsible for two-thirds of all patient cases [Apperley J. F., Lancet Oncology, 2007, 8, 1018-1029].

Soon afterwards, three 2^{nd} generation TKIs (namely nilotinib, dasatinib and bosutinib) were discovered to counter a number of those mutated BCR-ABL proteins. However, they are ineffective against the most prevalent point mutation, namely the T315I mutation at the gatekeeper region. The threonine (T) residue has been swapped by an isoleucine (I) residue, hence the important hydrogen bonding interactions by imatinib or these 2^{nd} generation TKI inhibitors that bind with threonine-315, can no longer bind effectively with the hydrophobic isoleucine motif.

To specifically target this T315I mutation, ponatinib was discovered by computer assisted molecular modellings and approved in 2012 for the treatment of refractory CML with Bcr-Abl (T315I) mutation. Despite its high potency against Bcr-Abl wild type, T315I mutants, and other point mutations, ponatinib can be very harmful to human health as it bears a black box warning. The use of ponatinib can cause serious vascular adverse events, which include a loss or severe narrowing of blood flow to the heart and brain, which may require emergency surgical intervention to restore blood flow. Arterial or venous thrombosis and occlusions, and heart failures can result in patient fatalities. Since majority of CML patients are older generation (>60 years old), with higher likelihood to suffer from cardiovascular disease, therefore prolonged treatment of ponatinib is extremely risky.

Hence, it is very important to identify novel TKI inhibitors that is as safe and effective as like imatinib, for CML patients with T315I mutation or other point mutations that 2^{nd} generation TKIs failed to be effective in the control of the disease.

### SUMMARY OF INVENTION

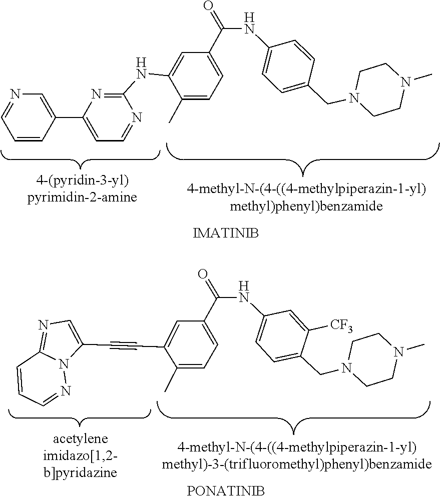 [SMILES: Cc1ccc(C(=O)Nc2ccc(CN3CCN(C)CC3)cc2)cc1Nc1nccc(-c2cccnc2)n1 ; Cc1ccc(C(=O)Nc2ccc(CN3CCN(C)CC3)c(C(F)(F)F)c2)cc1C#Cc1cnc2cccnn12]

To address on the difference in toxicity profiles between imatinib and ponatinib, it can somewhat be answered based on their chemical structures. On the right hand side of the chemical structure (see above), imatinib has a 4-methyl-N-(4-((4-methylpiperazin-1-yl)methyl)phenyl)benzamide fragment and ponatinib has a similar fragment, but bears an additional trifluoromethyl substituent at the 3 position of phenyl benzamide. However on the left hand side of those compounds, there are marked differences, where imatinib has a 4-(pyridin-3-yl)pyrimidin-2-amine fragment, but ponatinib has an acetylene imidazo[1,2-b]pyridazine fragment, so we could simply rationalize that the latter fragment has sole responsibility for the adverse cardiovascular events of ponatinib.

However, the cause of adverse events by ponatinib has not been proven but some researchers have postulated that toxicity can be due to off-target inhibitions of other kinases such as VEGFR 1-3 (receptor kinases known for the pathway to angiogenesis and vasculogenesis), or fibroblast growth factor receptors (FGFR) etc. In general, acetylene containing compounds are inherently known for the cause of toxicities due to the high reactivity of its carbon-carbon triple bonds that would led to a high risk of mechanism based inactivation of cytochrome P450 enzymes [Ortiz de Montellano P. R., et al., Drug Metabolism reviews, 2019, 162-177].

Herein we investigated on 1,3-disubstituted bicyclopentane (BCP) as novel kinase inhibitors for wild-type Bcr-Abl and mutations of Bcr-Abl.

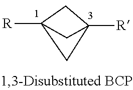

The BCP moiety was first reported in 1982 [Wiberg K. B., et al, J. Am. Chem. Soc., 1982, 104, 5239-5240] and a practical synthetic method of BCP-1,3-dicarboxylic acid was published in late 1980s [Michl J., et al, J. Org. Chem., 1988, 53, 4593-4596]. However, the use of this BCP moiety in drug discovery was very limited [Barbachyn M. R., et al, Bioorg. Med. Chem. Lett., 1993, 3, 671-676] [Pellicciari R., et al, J. Med. Chem. 1996, 39, 2874-2876], mainly due to the chemical synthetic methods for the construction of a BCP moiety into complex molecules were not well established at the time. More recently in 2012, BCP containing compounds are gamma-secretase inhibitors. [Stepan A. F., et al, J. Med. Chem, 2012, 55, 3414-3424].

### DETAILED DESCRIPTION OF INVENTION

The fusion protein BCR-Abl is a result of a reciprocal translocation that fuses the Abl proto-oncogene with the Bcr gene. BCR-Abl is then capable of transforming B-cells through the increase of mitogenic activity. This increase results in a reduction of sensitivity to apoptosis, as well as altering the adhesion and homing of CML progenitor cells. The present invention provides compounds, compositions and methods for the treatment of kinase related disease, particularly the Abl and Bcr-Abl, kinase related diseases. For example, leukemia and other proliferation disorders related to Bcr-Abl can be treated through the inhibition of wild type and mutant forms of Bcr-Abl.

The targeted TKI compounds have the following two scaffolds (Formula I and II).

In this disclosure, we report on novel class of TKI inhibitors of the structure form as shown in Formula I

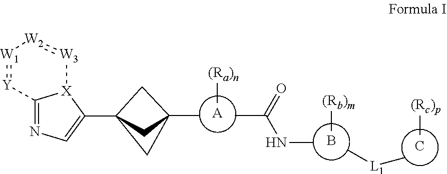

or a tautomer or an individual enantiomeric isomer thereof in which:
X is selected from NRₓ, O or S;
Rₓ is selected from H, C1-4 alkyl, C1-4 haloalkyl or a covalent bond, if X and Y together with the atoms which
X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
Ring A represents a 5- or 6 membered aryl or heteroaryl ring system, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a 5- or 6 membered aryl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);

L₁ is selected from —(CH₂)_{q}— (where q=1-2), O or a covalent bond;

Ring C represents a 5- or 6 membered aryl, heteroaryl, cycloalkyl or heterocycloalkyl and is substituted with R_{c} groups, where p=0-3;

R_{c}, at each occurrence, is independently selected from a group consisting of halogen, CH₃, CD₃, CF₃, C2-4 alkyl (branched or unbranched) or haloalkyl C1-4 (branched or unbranched).

And an alternative chemical scaffold that is a regioisomer of formula I, which is shown here as Formula II

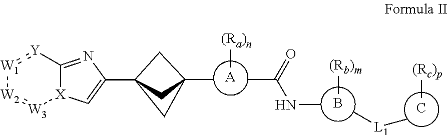

or a tautomer or an individual enantiomeric isomer thereof in which:
X is selected from NRₓ, O or S;
Rₓ is selected from H, C1-4 alkyl, C1-4 haloalkyl or a covalent bond, if X and Y together with the atoms which X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
Ring A represents a 5- or 6 membered aryl or heteroaryl ring system, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a 5- or 6 membered aryl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);

L₁ is selected from —(CH₂)_{q}— (where q=1-2), 0 or a covalent bond;

Ring C represents a 5- or 6 membered aryl, heteroaryl, cycloalkyl or heterocycloalkyl and is substituted with R_{c} groups, where p=0-3;

R_{c}, at each occurrence, is independently selected from a group consisting of halogen, CH₃, CD₃, CF₃, C2-4 alkyl (branched or unbranched) or haloalkyl C1-4 (branched or unbranched).

### Structural Definitions

“Alkyl” as a functional group with general formula CₙHₙ₊₂ and as a structural element of other groups, for example alkoxy, can be either branched or straight-chained (unbranched). C₁₋₄-alkoxy includes, methoxy, ethoxy, and the like. “Haloalkyl” means halogen-substituted alkyl includes trifluoromethyl, pentafluoroethyl, chloromethyl and the like.

“Aryl” means a monocyclic or fused bicyclic aromatic ring assembly containing six to ten ring carbon atoms. For example, aryl may be phenyl or naphthyl, preferably phenyl.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Halogen” (or halo) preferably represents chloro or fluoro, but may also be bromo or iodo.

“Heteroaryl” is as defined for aryl where one or more of the ring members are a heteroatom. For example heteroaryl includes pyridyl, pyrimidinyl, furanyl, oxazolyl, isoxazolyl, triazolyl, tetrazolyl, pyrazolyl, thienyl, indolyl, indazolyl, quinoxalinyl, quinolinyl, benzofuranyl, benzopyranyl, benzothiopyranyl, benzo[1,3]dioxole, imidazolyl, benzo-imidazolyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Treat”, “treating” and “treatment” refer to a method of alleviating or abating a disease and/or its attendant symptoms.

### Pharmacology and Utility

Compounds of the invention modulate the activity of protein tyrosine kinases and, as such, are useful for treating diseases or disorders in which protein tyrosine kinases, particularly the Abl, BCR-Abl, Bmx, CSK, TrkB, FGFR3, Fes, Lck, B-RAF, C-RAF, MKK6, SAPK2α and SAPK2β kinases, contribute to the pathology and/or symptomology of the disease.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) Bcr-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic Bcr-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T315I, F317L and M351T.

Compounds of the present invention inhibit abl kinase, especially v-abl kinase. The compounds of the present invention also inhibit wild-type Bcr-Abl kinase and mutations of Bcr-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

### Synthesis of Bromo-Ketone (15)

It is envisioned that the synthesis of bromoketone 15 as a key intermediate for the preparation of target compounds as shown as inhibitor series of Formula II (see scheme 1). Commercially available diketone 1 can be converted to the bromoketone 15 in 14 synthetic steps as depicted in Scheme 2. The bromoketone 15 would undergo a cyclization to construct the heteroaromatic ring system which upon direct hydrolysis of the ethyl benzoate ester to the corresponding carboxylic acid, and then subsequent amide coupling reaction to afford a series of target compounds of interest. Our synthetic approach in the preparation of target compounds do not limited to this route that is described herein.

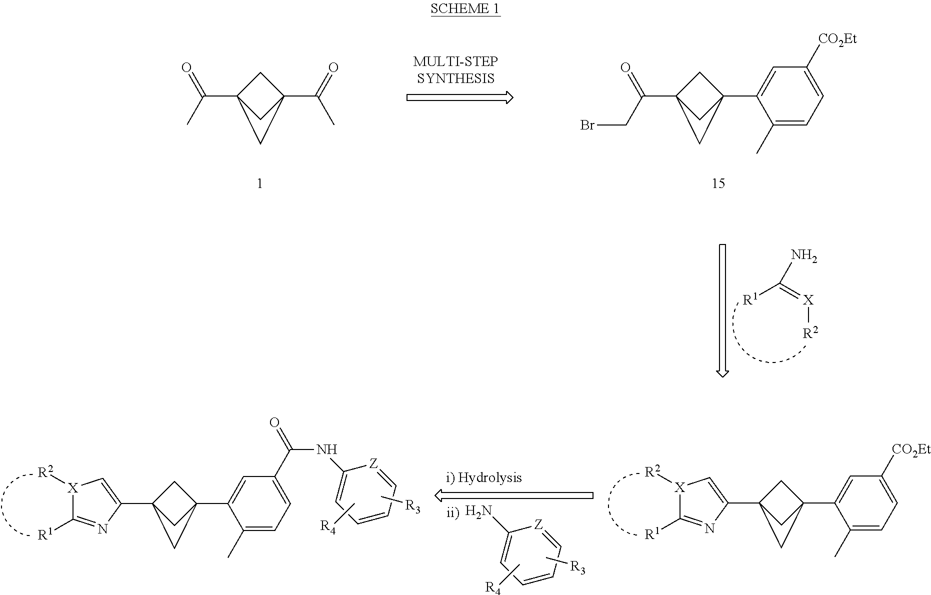

### Experimental Procedure for the Preparation of Intermediate Bromo-Ketone (15)

Compound 1 can be prepared by following reference literature. [Michl J., et al, J. Org. Chem, 1988, 53, 4593-4594]

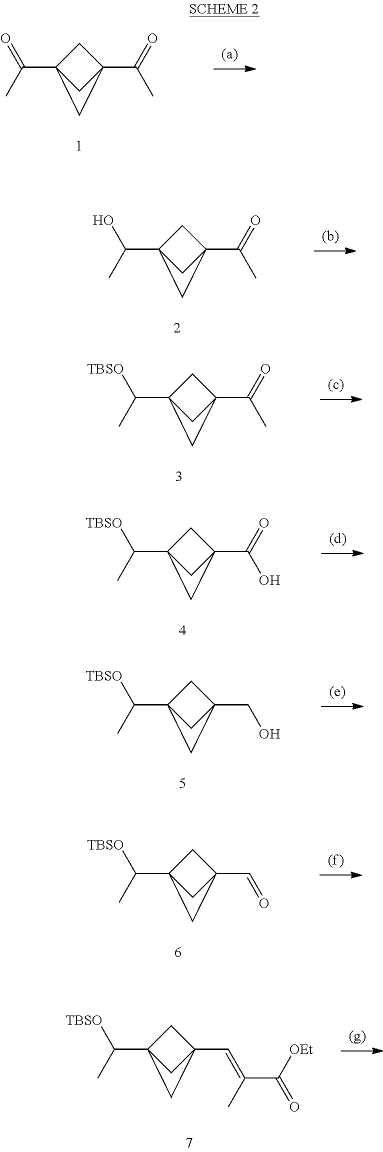

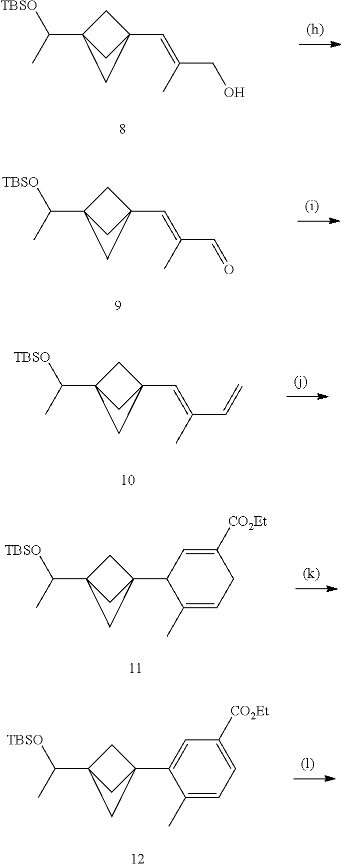

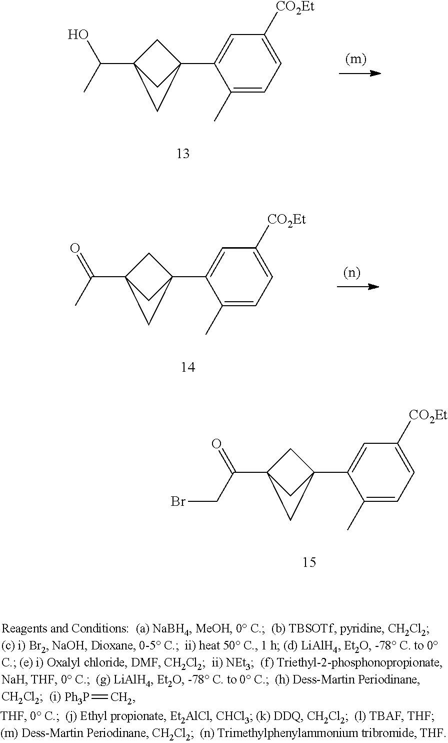

### Preparation of 1-(3-(1-hydroxyethyl)bicyclo[1.1.1]pentan-1-yl)ethan-1-one (2)

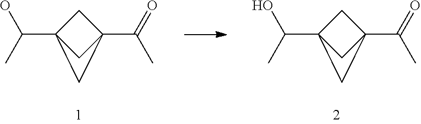

To a solution of diketone 1 (MW 152, 3.70 g, 24.3 mmol) in 28 ml of methanol was added sodium borahydride (MW 38, 220 mg, 5.79 mmol) in portion at ice-bath temperature over a period of 20 mins.

Afterwards, the reaction mixture was allowed to stir for addition 1 h at room temperature before quenching with 2 mL solution of sodium bicarbonate at 5° C. The reaction mixture was concentrated in vacuo to afford the crude product, which was extracted with ethyl acetate, the organic layers were combined and dried over sodium sulfate, filtered and concentrated in vacuo. The material was purified by silica gel chromatography with 10-30% Ethyl acetate/Hexanes to afford the hydroxyketone 2 as a colorless oil (1.94 g, 12.6 mmol, 51% yield). The diol was isolated and starting material, diketone, was recovered from this reaction.

¹HNMR (500 MHz, CDCl₃) δ ppm 4.75 (q, 1H), 2.06 (s, 3H), 1.87 (d, 3H), 1.82 (d, 3H), 1.50 (bs, 1H), 1.07 (d, 3H).

### Preparation of 1-(3-(1-((tert-butyldimethylsilyl)oxy)ethyl)bicyclo[1.1.1]pentan-1-yl)ethan-1-one (3)

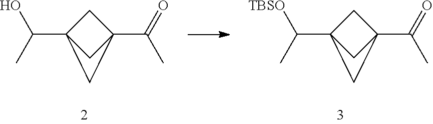 [SMILES: CC(=O)C12CC(C(C)O)(C1)C2 ; CC(=O)C12CC(C(C)O[Si](C)(C)C(C)(C)C)(C1)C2]

To a solution of hydroxyketone 2 (MW 154, 1.94 mg, 12.6 mmol) and pyridine (MW 79, d 0.982, 2 ml, 24.9 mmol) in 20 ml of dichloromethane was added a solution of tert-butyldimethylsilyl triflate (MW 264, d 1.15, 3.2 mL, 13.9 mmol) in dichloromethane (2 mL) at −78° C. and stirred for 1 h. The reaction mixture was allowed to stir at ice bath temperature for addition 1 h, before quenching with 2 mL solution of sodium bicarbonate at ice bath temperature. Crude product was extracted with diethyl ether (X2), the organic layers were combined and dried over sodium sulfate, filtered and concentrated in vacuo, which was purified by silica gel chromatography with 5-10% Ethyl acetate/Hexanes to afford the TBS protected ether 3 as a colorless oil (3.2 g, 11.9 mmol, 95% yield).

### Preparation of 3-(1-((tert-butyldimethylsilyl)oxy)ethyl)bicyclo[1.1.1]pentane-1-carboxylic Acid (4)

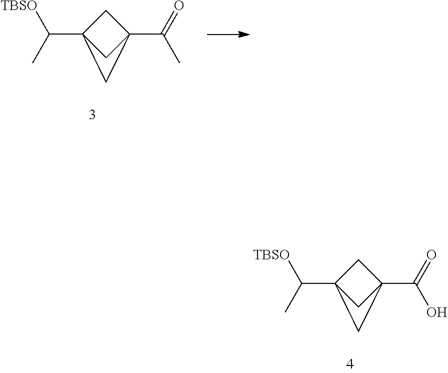 [SMILES: CC(=O)C12CC(C(C)O[Si](C)(C)C(C)(C)C)(C1)C2 ; CC(O[Si](C)(C)C(C)(C)C)C12CC(C(=O)O)(C1)C2]

To a solution of sodium hydroxide in water (50 mL) and 1,4-dioxane (10 mL) of was added bromine (MW 160, 8.31 g, 52.0 mmol) over 10 mins at ice-bath temperature. To this resulting yellow solution of sodium hypobromite, was added a solution of TBS-protected ketone 3 (MW 268, 3.2 g, 11.9 mmol) in 1,4-dioxane (10 mL) at 1-5° C., over the a period of 2 hrs. Afterwards, the reaction mixture was allowed to stir at ice bath temperature for addition 1 h, and then warmed up to room temperature stirred for another 3 hr, and heated at 50° C. for 1 hr. It was extracted with ethyl acetate (X3), the organic layers were combined and dried over sodium sulfate, filtered and concentrated in vacuo, to afford the crude TBS protected acid 4 as a colorless waxy solid (3.3 g).

### Preparation of (3-(1-((tert-butyldimethylsilyl)oxy)ethyl)bicyclo[1.1.1]pentan-1-yl)methanol (5)

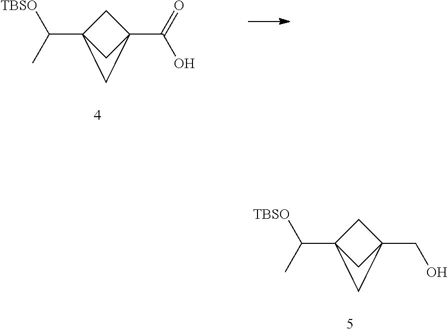 [SMILES: CC(O[Si](C)(C)C(C)(C)C)C12CC(C(=O)O)(C1)C2 ; CC(O[Si](C)(C)C(C)(C)C)C12CC(CO)(C1)C2]

To a solution of crude TBS protected carboxylic acid 4 (MW 270, 3.3 mg) in 30 ml of diethyl ether was added 1.0 M solution of lithium aluminium hydride (3.2 mL, 3.2 mmol) dropwise at −78° C. The reaction mixture was allowed to stir at ice bath temperature for addition 2 h, before quenching with 2 mL solution of sodium bicarbonate at −50° C. It was repeatedly extracted five times with ethyl acetate, the organic layers were combined and dried over sodium sulfate, filtered and concentrated in vacuo to afford the crude product, which was purified by silica gel chromatography with 25% Ethyl acetate/Hexanes to afford the TBS protected alcohol 5, as a colorless oil (1.95 g, 7.6 mmol, 64% yield over 2 steps from the hydroxyketone).

### Preparation of 3-(1-((tert-butyldimethylsilyl)oxy)ethyl)bicyclo[1.1.1]pentane-1-carbaldehyde (6)

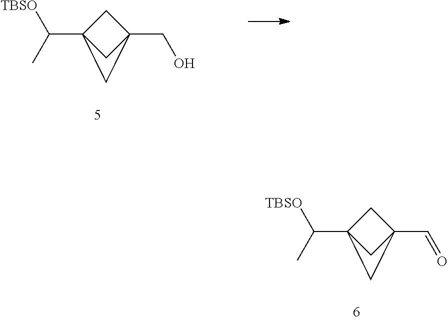 [SMILES: CC(O[Si](C)(C)C(C)(C)C)C12CC(CO)(C1)C2 ; CC(O[Si](C)(C)C(C)(C)C)C12CC(C=O)(C1)C2]

To a solution of oxalyl chloride (MW 127, 2.0 g, 15.7 mmol) in 30 mL of dichloromethane was added dimethylsulfoxide (FW78, 1.1 mL, 15.5 mmol) dropwise at −78° C. and stirred for 15 minutes. Afterwards, TBS-protected alcohol 5 (MW 256, 1.95 g, 7.6 mmol) in 2 ml of dichloromethane was added dropwise −78° C. and stirred for 20 mins. Subsequently, triethylamine (MW 101, d 0.726, 3.2 ml, 23.3 mmol) was added and the reaction mixture was allowed to stir at ice bath temperature for 30 mins, before quenching with 2 mL solution of sodium bicarbonate. Crude product was extracted with diethyl ether (X2), the organic layers were combined and dried over sodium sulfate, filtered and concentrated in vacuo, to afford the crude aldehyde 6 (2.0 g), as a yellow orange oil, which was used in the next step without further purification.

### Preparation of ethyl (E)-3-(3-(1-((tert-butyldimethylsilyl)oxy)ethyl)bicyclo[1.1.1]pentan-1-yl)-2-methylacrylate (7)

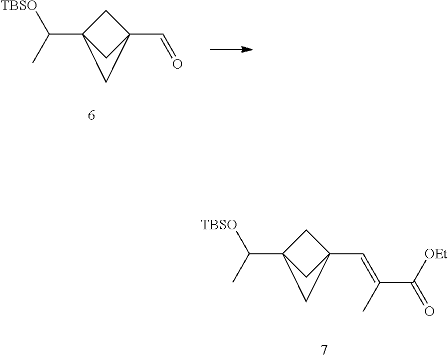 [SMILES: CC(O[Si](C)(C)C(C)(C)C)C12CC(C=O)(C1)C2 ; CCOC(=O)/C(C)=C/C12CC(C(C)O[Si](C)(C)C(C)(C)C)(C1)C2]

To a solution of crude aldehyde 6 (MW 254, 2.0 g, ˜7.6 mmol) and triethyl-2-phosphonopropionate (MW 238, 2.32 g, 9.74 mmol) in 20 ml of dry tetrahydrofuran was added 60% sodium hydride in mineral oil (MW 24, 464 mg, 11.6 mmol) at ice bath temperature and the resulting reaction mixture was allowed to stir at room temperature for 2 h. Afterwards, the reaction was quenched with 2 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 5-15% ethyl acetate/hexanes to afford the ethyl ester 7 as a colorless oil (1.93 g, 5.71 mmol, 75% yield over 2 steps from the alcohol).

### Preparation of (E)-3-(3-(1-((tert-butyldimethylsilyl)oxy)ethyl)bicyclo[1.1.1]pentan-1-yl)-2-methylprop-2-en-1-ol (8)

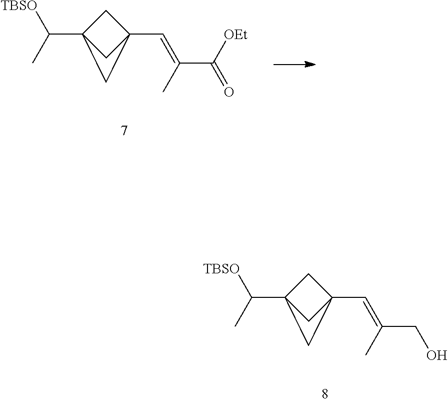 [SMILES: CCOC(=O)/C(C)=C/C12CC(C(C)O[Si](C)(C)C(C)(C)C)(C1)C2 ; C/C(=C\C12CC(C(C)O[Si](C)(C)C(C)(C)C)(C1)C2)CO]

To a solution of ethyl ester 7 (MW 338, 1.93 g, 5.71 mmol) in 10 ml of diethyl ether was added 1M solution of diisobutylaluminium hydride (DIBAL-H, 14.0 mL, 14.0 mmol) dropwise at −78° C. and stirred for 1 h, before quenching with solution of sodium bicarbonate. Crude product was extracted with diethyl ether (X2), the organic layers were combined and dried over sodium sulfate, filtered and concentrated in vacuo, which was purified by silica gel chromatography with hexanes and 25% Ethyl acetate/hexanes to afford the allylic alcohol 8, as a colorless oil (1.33 g, 4.49 mmol, 78% yield).

### Preparation of (E)-3-(3-(1-((tert-butyldimethylsilyl)oxy)ethyl)bicyclo[1.1.1]pentan-1-yl)-2-methylacrylaldehyde (9)

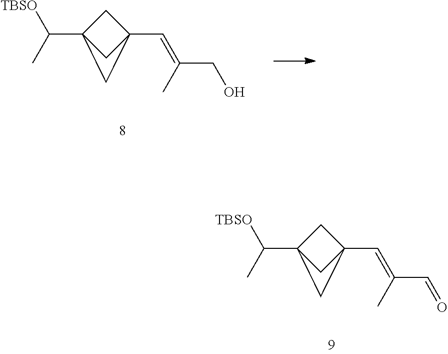 [SMILES: C/C(=C\C12CC(C(C)O[Si](C)(C)C(C)(C)C)(C1)C2)CO ; C/C(C=O)=C\C12CC(C(C)O[Si](C)(C)C(C)(C)C)(C1)C2]

To a solution of alcohol 8 (MW 338, 1.33 g, 4.49 mmol) in 30 ml of dry dichloromethane was added Dess-Martin periodinane (MW 424.14, 2.14 g, 5.04 mmol) at ice bath temperature and the resulting reaction mixture was allowed to stir at room temperature for 2 h. Afterwards, the reaction was quenched with 2 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 20% Ethyl acetate/Hexanes to afford the crude aldehyde 9, as a colorless oil (1.15 g, 3.91 mmol, 87% yield). The material was used in the next step without further purification.

### Preparation of (E)-tert-butyldimethyl(1-(3-(2-methylbuta-1,3-dien-1-yl)bicyclo[1.1.1]pentan-1-yl)ethoxy)silane (10)

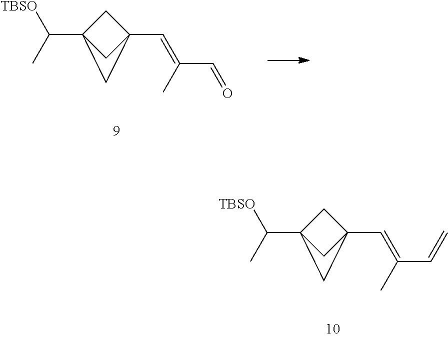 [SMILES: C/C(C=O)=C\C12CC(C(C)O[Si](C)(C)C(C)(C)C)(C1)C2 ; C=C/C(C)=C/C12CC(C(C)O[Si](C)(C)C(C)(C)C)(C1)C2]

To a solution of conjugated aldehyde 9 (MW 294, 1.15 g, 3.91 mmol) in 30 ml of dry diethyl ether was added 0.25 M solution of methylenetriphenylphosphorane CAS 3487-44-3 (18.0 mL, 4.5 mmol) dropwise at −78° C. The reaction mixture was allowed to stir at ice bath temperature for addition 1 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of hexanes and 5% Ethyl acetate/Hexanes to afford the alkene 10, as a colorless oil (0.83 g, 2.84 mmol, 72% yield).

¹HNMR (500 MHz, CDCl₃) δ ppm 6.57 (dd, 1H), 5.43 (s, 1H), 5.08 (d, 1H), 4.91 (d, 1H), 3.45 (q, 1H), 1.79 (d, 3H), 1.77 (s, 3H), 1.74 (d, 3H), 1.02 (d, 3H), 0.85 (s, 9H), 0.00 (s, 6H).

### Preparation of ethyl 3-(3-(1-((tert-butyldimethylsilyl)oxy)ethyl)bicyclo[1.1.1]pentan-1-yl)-4-methylcyclohexa-1,4-diene-1-carboxylate (11)

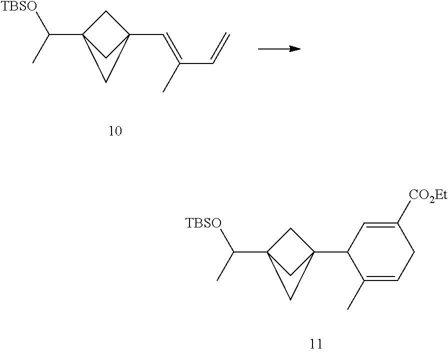 [SMILES: C=C/C(C)=C/C12CC(C(C)O[Si](C)(C)C(C)(C)C)(C1)C2 ; CCOC(=O)C1=CC(C23CC(C(C)O[Si](C)(C)C(C)(C)C)(C2)C3)C(C)=CC1]

To a solution of diene 10 (MW 292, 400 mg, 1.36 mmol) and ethyl propiolate (MW 98, 225 mg, 2.29 mmol) in 8 ml of chloroform was added 1.0 M solution of diethylaluminium chloride CAS (MW 120.56, 0.7 mL, 0.7 mmol) and allow to stir at room temperature for 24 h. Afterwards, the reaction was quenched with 2 mL solution of sodium bicarbonate. Crude product was extracted with ethyl acetate (X2), the organic layers were combined and dried over sodium sulfate, filtered and concentrated in vacuo, to afford the crude diene 11 (340 mg, 0.87 mmol, 64% yield). The material was used in the next step without further purification.

### Preparation of ethyl 3-(3-(1-((tert-butyldimethylsilyl)oxy)ethyl)bicyclo[1.1.1]pentan-1-yl)-4-methylbenzoate (12)

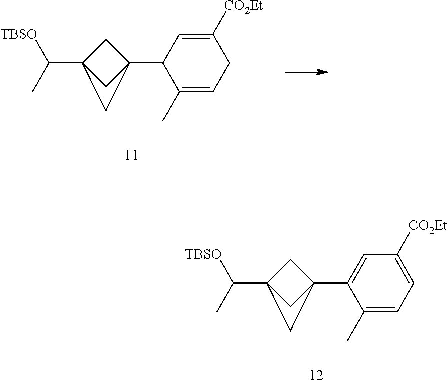 [SMILES: CCOC(=O)C1=CC(C23CC(C(C)O[Si](C)(C)C(C)(C)C)(C2)C3)C(C)=CC1 ; CCOC(=O)c1ccc(C)c(C23CC(C(C)O[Si](C)(C)C(C)(C)C)(C2)C3)c1]

To a solution of crude diene 11 (MW 390, 340 mg, 0.87 mmol) in 20 ml of dichloromethane was added 2,3-dichloro-5,6-dicyano-1,4-benzoquinone (DDQ, MW 231, 220 mg, 0.95 mmol) at room temperature. The reaction mixture turn dark immediately and it was allowed to stir for 2 h. Afterwards, solution of sodium bicarbonate was added and the crude product was extracted with diethyl ether (X2), the organic layers were combined and dried over sodium sulfate, filtered and concentrated in vacuo. The material was purified by silica gel chromatography with elution of hexanes and 5% ethyl acetate/hexanes to afford the alkene 12, as a colorless oil (285 mg, 0.73 mmol, 85% yield).

¹HNMR (500 MHz, CDCl₃) δ ppm 7.70 (m, 2H), 7.20 (d, 1H), 4.29 (q, 2H), 3.70 (q, 1H), 2.37 (s, 3H), 1.98 (d, 3H), 1.92 (d, 3H), 1.31 (q, 3H), 1.06 (d, 3H), 0.84 (s, 9H), 0.00 (s, 6H)

### Preparation of ethyl 3-(3-(1-hydroxyethyl)bicyclo[1.1.1]pentan-1-yl)-4-methylbenzoate (13)

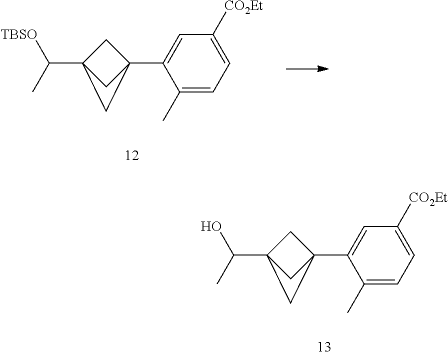 [SMILES: CCOC(=O)c1ccc(C)c(C23CC(C(C)O[Si](C)(C)C(C)(C)C)(C2)C3)c1 ; CCOC(=O)c1ccc(C)c(C23CC(C(C)O)(C2)C3)c1]

To a solution of TBS-protected ether 12 (MW 388, 275 mg, 0.71 mmol) in 5 ml of tetrahydrofuran was added 1.0 M solution of tetra-n-butylammonium fluoride (1.0 mL, 1.0 mmol) at −20° C. The reaction mixture was allowed to stir at room temperature for addition 24 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of 5-25% Ethyl acetate/Hexanes to afford the alcohol 13, as a colorless oil (170 mg, 0.62 mmol, 87% yield).

### Preparation of ethyl 3-(3-acetylbicyclo[1.1.1]pentan-1-yl)-4-methylbenzoate (14)

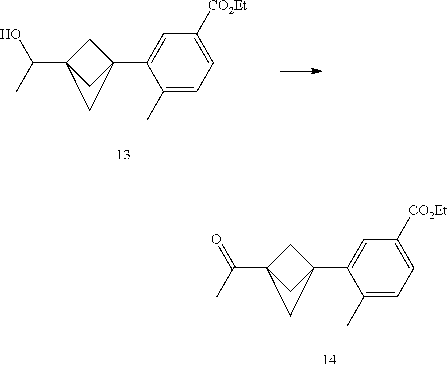 [SMILES: CCOC(=O)c1ccc(C)c(C23CC(C(C)O)(C2)C3)c1 ; CCOC(=O)c1ccc(C)c(C23CC(C(C)=O)(C2)C3)c1]

To a solution of alcohol 13 (MW 274, 170 mg, 0.62 mmol) in 5 ml of dry dichloromethane was added Dess-Martin periodinane (MW 424.14, 285 mg, 0.67 mmol) at ice bath temperature and the resulting reaction mixture was allowed to stir at room temperature for 2 h. Afterwards, the reaction was quenched with 2 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 10% Ethyl acetate/Hexanes to afford the ketone 14, as a colorless oil (136 mg, 0.50 mol, 80% yield).

### Preparation of ethyl 3-(3-(2-bromoacetyl)bicyclo[1.1.1]pentan-1-yl)-4-methylbenzoate (15)

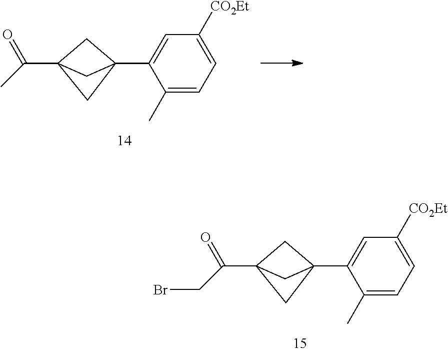 [SMILES: CCOC(=O)c1ccc(C)c(C23CC(C(C)=O)(C2)C3)c1 ; CCOC(=O)c1ccc(C)c(C23CC(C(=O)CBr)(C2)C3)c1]

To a solution of crude aldehyde 14 (MW 272, 136 mg, 0.50 mmol) in 3 ml of dry THF was added phenyltrimethylammonium tribromide (MW 376, 189 mg, 0.50 mmol) at room temperature, and it was allowed to stir at room temperature for 2 h. Afterwards, the reaction mixture was quenched with 2 mL solution of sodium bicarbonate. Crude product was extracted with diethyl ether (X2), the organic layers were combined and dried over sodium sulfate, filtered and concentrated in vacuo, to afford the crude bromide 15, as a brownish oil (220 mg). The material was used in the next step without further purification.

### Synthesis of Bromo-Aldehyde (35)

It is envisioned that the synthesis of bromoaldehyde 35 as a key intermediate for the preparation of target compound as shown in scheme 3. Commercially available diketone 1 can be converted to the ethyl diester 16 in two steps following the literature procedure. Bromoaldehyde can be prepared in 19 steps synthesis as depicted in scheme 4. Similar to the bromoketone 15 as in scheme 1, the bromoaldehyde 35 would undergo similar cyclization to construct the other regioisomer of the heteroaromatic ring system, which upon direct hydrolysis of the ethyl benzoate ester to the corresponding carboxylic acid and subsequent amide coupling reaction to afford a series of target compounds of interest. Our synthetic approach in the preparation of target compounds do not limited to this route that is described herein.

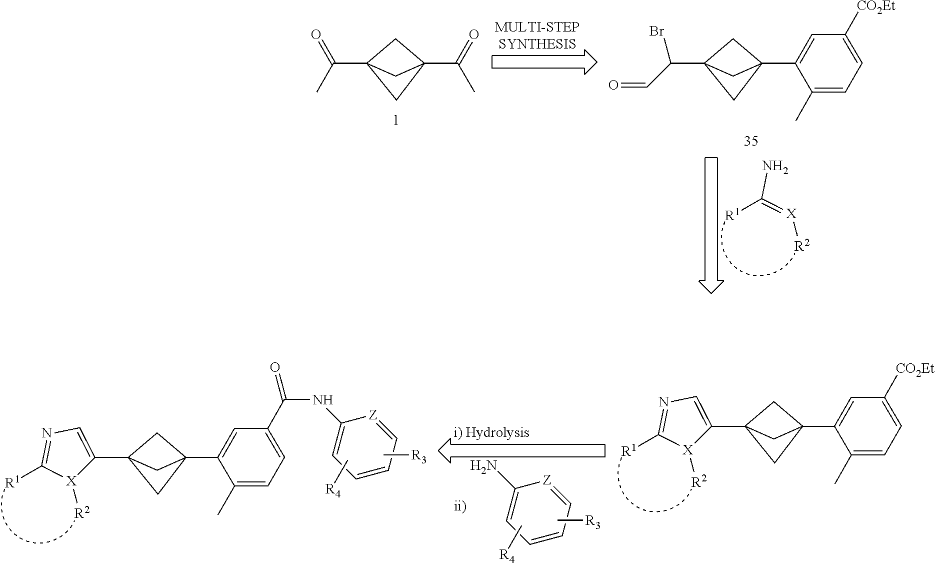

### Preparation of Intermediate Bromo-Aldehyde (35)

Compound 16 can be prepared from compound 1 by following reference literatures. [Michl J., et al., J. Org. Chem, 1988, 53, 4593-4594] [Pellicciari R., et al., J. Med. Chem, 1996, 39, 2874-2876]

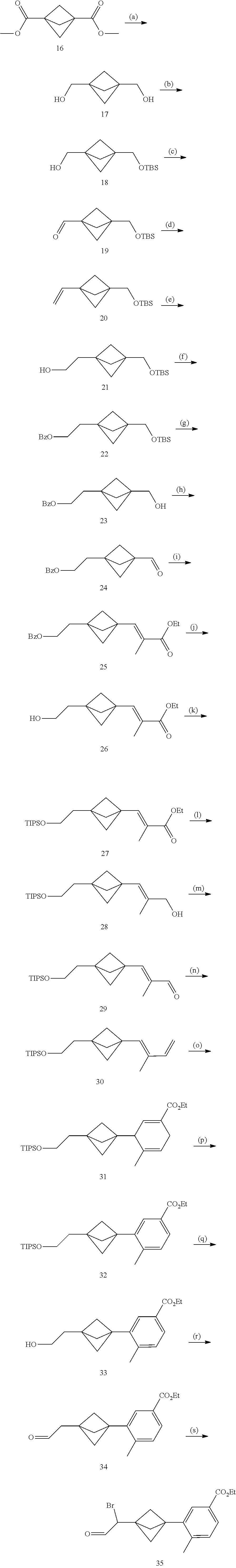

Reagents and Conditions: (a) LiAlH₄, Et₂O, −78° C.; (b) TBSCl, pyridine, CH₂Cl₂; (c) i) DMF, Oxalyl chloride, CH₂Cl₂, −78° C. to 0° C.; ii) NEt₃; (d) Ph₃P=CH₂, THF, −78° C. to 0° C.; (e) i) 9-BBN, THF, −78° C. to 0° C.; ii) NaOH, H₂O₂; (f) BzCl, pyridine, CH₂Cl₂, −78° C. to 0° C.; (g) TBAF, THF, −20° C. to RT; (h) Dess-Martin Periodinane, CH₂Cl₂; (i) Triethyl-2-phosphonopropionate, NaH, THF, 0° C.; (j) EtOK, ethanol, 0° C.; (k) TIPSCl, pyridine, CH₂Cl₂, −20° C. to RT; (1) LiAlH₄, Et₂O, −78° C. to 0° C.; (m) Dess-Martin Periodinane, CH₂Cl₂; (n) Ph₃P=CH₂, THF, −78° C. to 0° C.; (o) Ethyl propiolate, Et₂AlCl, Chloroform; (p) DDQ, CH₂Cl₂; (q) TBAF, THF, −20° C. to RT; (r) Dess-Martin Periodinane, CH₂Cl₂; (s) Phenyltrimethylammonium tribromide, THF.

### Scheme 4

### Experimental Procedure for the Preparation of Bromo-Aldehyde Intermediate

### Preparation of bicyclo[1.1.1]pentane-1,3-diyldimethanol (17)

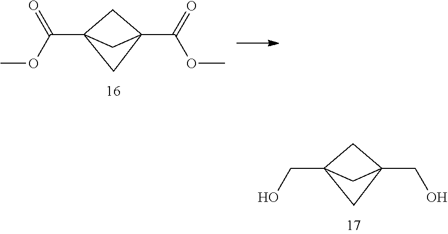

To a solution of diethyl ester 16 (MW 220, 2.04 g, 9.27 mmol) in 40 ml of diethyl ether was added 1.32 M solution of lithium aluminium hydride (10 mL, 1.32 mmol) dropwise at −78° C. The reaction mixture was allowed to stir at ice bath temperature for addition 2 h, before quenching with 3 mL solution of sodium bicarbonate at −50° C. It was repeatedly extracted five times with ethyl acetate, the organic layers were combined and dried over sodium sulfate, filtered and concentrated in vacuo to afford the crude product, which was purified by silica gel chromatography with 50% Ethyl acetate/Hexanes to afford the diol 17, as a colorless oil (1.05 g, 8.20 mmol, 88% yield).

¹HNMR 600 MHz (CDCl₃) δ 3.50 (s, 4H); 2.50 (s, 6H), 2.2 (bs, 2H).

### Preparation of (3-(((tert-butyldimethylsilyl)oxy)methyl)bicyclo[1.1.1]pentan-1-yl)methanol (18)

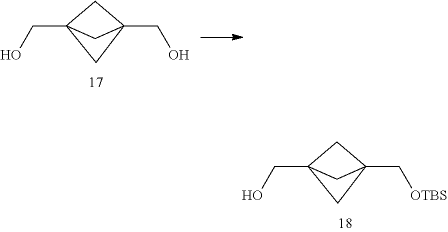 [SMILES: OCC12CC(CO)(C1)C2 ; CC(C)(C)[Si](C)(C)OCC12CC(CO)(C1)C2]

To a solution of diol 17 (MW 128, 800 mg, 6.25 mmol) and pyridine (2.0 ml, mmol) in 10 ml of dichloromethane was added a solution of tert-butyldimethylsilyl chloride (MW 156.5, 970 mg, 6.20 mmol) in dichloromethane (18 mL) at −20° C. and stirred for 1 h. The reaction mixture was allowed to stir at room temperature for addition 3 h, before quenching with 4 mL solution of sodium bicarbonate at ice bath temperature. Crude product was extracted with ethyl acetate (X3), the organic layers were combined and dried over sodium sulfate, filtered and concentrated in vacuo, which was purified by silica gel chromatography with 5-20% Ethyl acetate/Hexanes to afford the mono protected alcohol 18, as a colorless oil (0.58 g, 2.40 mmol, 38% yield). Bis protected ether was isolated and starting material, diol, was recovered from this reaction.

### Preparation of 3-(((tert-butyldimethylsilyl)oxy)methyl)bicyclo[1.1.1]pentane-1-carbaldehyde (19)

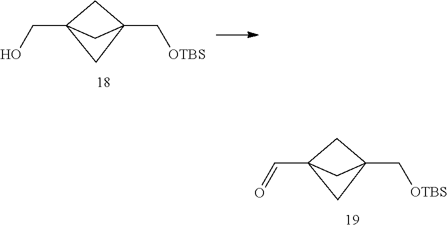 [SMILES: CC(C)(C)[Si](C)(C)OCC12CC(CO)(C1)C2 ; CC(C)(C)[Si](C)(C)OCC12CC(C=O)(C1)C2]

To a solution of oxalyl chloride (MW 127, 350 mg, 2.76 mmol) in 8.0 mL of dichloromethane was added dimethylsulfoxide (MW 78, 0.2 ml, d 1.101, 2.82 mmol) dropwise at −78° C. and stirred for 15 minutes. Afterwards, TBS-protected alcohol 18 (MW 242, 400 mg, 1.65 mmol) in 2.0 ml of dichloromethane was added dropwise −78° C. and stirred for 20 mins. Subsequently, triethylamine (1.0 ml, d 0.76, 7.52 mmol) was added and the reaction mixture was allowed to stir at ice bath temperature for 30 mins, before quenching with 2 mL solution of sodium bicarbonate. Crude product was extracted with diethyl ether (X2), the organic layers were combined and dried over sodium sulfate, filtered and concentrated in vacuo, to afford the crude aldehyde 19 (approx. 400 mg) as a yellow oil, which was used in the next step without further purification.

### Preparation of tert-butyldimethyl((3-vinylbicyclo[1.1.1]pentan-1-yl)methoxy)silane (20)

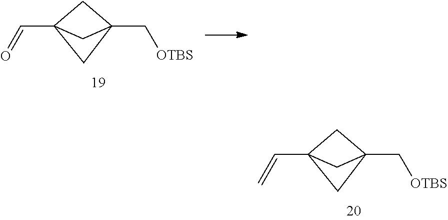 [SMILES: CC(C)(C)[Si](C)(C)OCC12CC(C=O)(C1)C2 ; C=CC12CC(CO[Si](C)(C)C(C)(C)C)(C1)C2]

To a solution of TBS-protected aldehyde 19 (MW 240, crude material 350 mg, approx. 1.45 mmol) in 20 ml of dry diethyl ether was added 0.23 M solution of methylenetriphenylphosphorane CAS 3487-44-3 (8.0 mL, 1.84 mmol) dropwise at −78° C. The reaction mixture was allowed to stir at ice bath temperature for addition 1 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of hexanes and 5% Ethyl acetate/Hexanes to afford the alkene 20, as a colorless oil (278 mg, 1.17 mmol, 80% yield).

### Preparation of 2-(3-(((tert-butyldimethylsilyl)oxy)methyl)bicyclo[1.1.1]pentan-1-yl)ethan-1-ol (21)

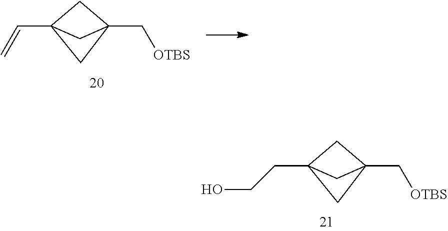 [SMILES: C=CC12CC(CO[Si](C)(C)C(C)(C)C)(C1)C2 ; CC(C)(C)[Si](C)(C)OCC12CC(CCO)(C1)C2]

To a solution of alkene 20 (MW 238, 2.05 g, 8.61 mmol) in 30 mL of dry tetrahydrofuran was added 0.65 M solution of 9-borabicyclo[3.3.1]nonane (9-BBN, 14.0 mL, 9.10 mmol) dropwise at −78° C. The reaction mixture was allowed to stir at ice bath temperature for addition 1 h, before addition of 0.5 ml of 37% solution hydrogen peroxide and 0.5 ml of 1.0 M solution of sodium hydroxide. After stirred at room temperature for 30 mins, 2 mL solution of sodium bicarbonate was added to quench the reaction. Crude product was extracted with diethyl ether (X2), the organic layers were combined and dried over sodium sulfate, filtered and concentrated in vacuo, which was purified by silica gel chromatography with elution of 10-25% Ethyl acetate/Hexanes to afford the primary alcohol 21, as a colorless oil (1.45 g, 5.66 mmol, 66% yield).

### Preparation of 2-(3-(((tert-butyldimethylsilyl)oxy)methyl)bicyclo[1.1.1]pentan-1-yl)ethyl benzoate (22)

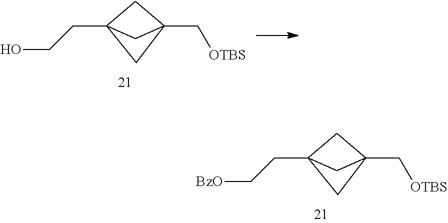

To a solution of alcohol 21 (MW 256, 3.35 g, 13.1 mmol) and pyridine (MW 79, d 0.982, 2.0 mL) in 30 ml of dry dichloromethane was added benzoyl chloride (MW 140.5, 2.25 mL, 19.4 mmol) dropwise at −78° C. The reaction mixture was allowed to stir at ice bath temperature for addition 1 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of hexanes and 5% Ethyl acetate/Hexanes to afford the benzoate ester 22, as a colorless oil (3.8 g, 10.5 mmol, 81% yield).

### Preparation of 2-(3-(hydroxymethyl)bicyclo[1.1.1]pentan-1-yl)ethyl benzoate (23)

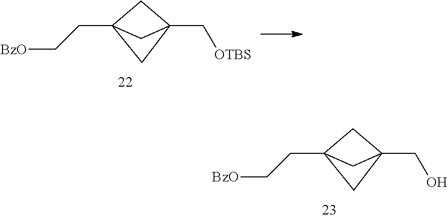

To a solution of TBS-protected ether 22 (MW 360, 3.7 g, 10.3 mmol) in 20 ml of tetrahydrofuran was added 1.0 M solution of tetra-n-butylammonium fluoride (20.0 mL, 20 mmol) at −20° C. The reaction mixture was allowed to stir at room temperature for addition 4 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of 5-25% Ethyl acetate/Hexanes to afford the alcohol 23, as a colorless oil (2.3 g, 9.3 mmol, 91% yield).

### Preparation of 2-(3-formylbicyclo[1.1.1]pentan-1-yl)ethyl benzoate (24)

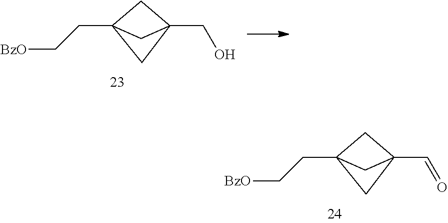

To a solution of alcohol 23 (MW 246, 1.60 g, 6.5 mmol) in 50 ml of dry dichloromethane was added Dess-Martin periodinane (MW 424.14, 3.0 g, 7.1 mmol) at ice bath temperature and the resulting reaction mixture was allowed to stir at room temperature for 2 h. Afterwards, the reaction was quenched with 10 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 20% Ethyl acetate/Hexanes to afford the aldehyde 24, as a colorless oil (1.40 g, 5.7 mmol, 88% yield). The material was used in the next step without further purification.

### Preparation of (E)-2-(3-(3-ethoxy-2-methyl-3-oxoprop-1-en-1-yl)bicyclo[1.1.1]pentan-1-yl)ethyl benzoate (25)

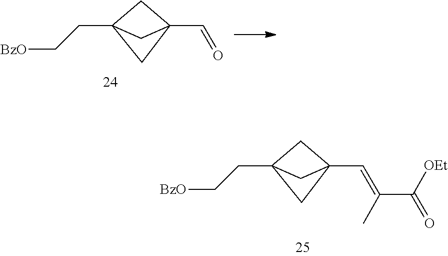

To a solution of aldehyde 24 (MW 244, 760 mg, mmol) and triethyl-2-phosphonopropionate (MW 238, 809 mg, 3.4 mmol) in 20 ml of dry tetrahydrofuran was added 60% sodium hydride in mineral oil (MW 24, 140 mg, ˜3.5 mmol) at ice bath temperature and the resulting reaction mixture was allowed to stir at room temperature for 2 h. Afterwards, the reaction was quenched with 2 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 5-15% ethyl acetate/hexanes to afford the ethyl ester 25, as a colorless oil (810 mg, 80% yield).

¹HNMR (500 MHz, CDCl₃) δ ppm 7.95 (d, J=8.0 Hz, 1H), 7.49 (t, J=8.0 Hz, 1H), 7.37 (t, J=8.0 Hz, 1H), 6.57 (s, 1H), 4.27 (t, J=6.0 Hz, 1H), 4.08 (q, J=7.0 Hz, 2H), 1.90 (s, 6H), 1.88 (t, J=7.0 Hz), 1.81 (s, 3H), 1.20 (t, J=7.0 Hz, 3H).

### Preparation of ethyl (E)-3-(3-(2-hydroxyethyl)bicyclo[1.1.1]pentan-1-yl)-2-methylacrylate (26)

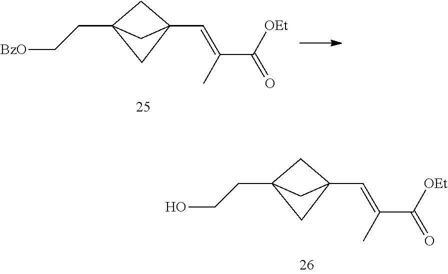

To a solution of benzoate ester 25 (MW 328, 800 mg, 2.4 mmol) in 10 ml of dry ethanol and 10 ml of dry tetrahydrofuran was added 1.0 M solution of potassium ethoxide in ethanol (5.0 mL, 5.0 mmol) at ice bath temperature. The reaction mixture was allowed to stir at room temperature for addition 4 h, before quenching with 0.2 mL solution of glacial acetic acid and water. Crude product was extracted with diethyl ether (X2), the organic layers were combined and dried over sodium sulfate, filtered and concentrated in vacuo, which was purified by silica gel chromatography with elution of 10-25% ethyl acetate/hexanes to afford the primary alcohol 26, as a colorless oil (520 mg, 95% yield).

### Preparation of ethyl (E)-2-methyl-3-(3-(2-((triisopropylsilyl)oxy)ethyl)bicyclo[1.1.1]pentan-1-yl)acrylate (27)

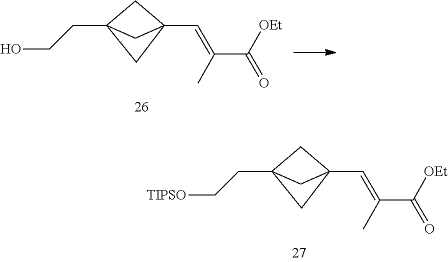

To a solution of alcohol 26 (MW 224, 567 mg, 2.53 mmol) and pyridine (MW 78, 1.0 g, 12.8 mmol) in 20 ml of dichloromethane was added a solution of triisopropylsilyl chloride (MW 306.4, 860 mg, mmol) in dichloromethane (2 mL) at −20° C. and stirred for 1 h. The reaction mixture was allowed to stir at room temperature for addition 3 h, before quenching with 2 mL solution of sodium bicarbonate at ice bath temperature. Crude product was extracted with diethyl ether (X2), the organic layers were combined and dried over sodium sulfate, filtered and concentrated in vacuo, which was purified by silica gel chromatography with hexanes and 5% Ethyl acetate/hexanes to afford the TIPS protected ether 27, as a colorless oil (750 mg, 1.97 mmol, 78% yield).

### Preparation of (E)-2-methyl-3-(3-(2-((triisopropylsilyl)oxy)ethyl)bicyclo[1.1.1]pentan-1-yl)prop-2-en-1-ol (28)

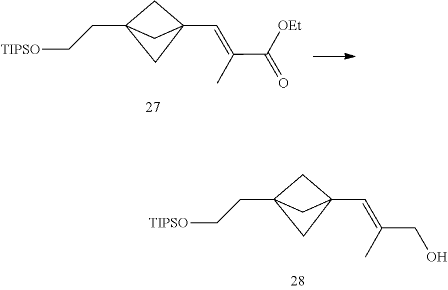

To a solution of ethyl ester 27 (MW 380, 740 mg, 1.94 mmol) in 10 ml of diethyl ether was added 1.0 M solution of lithium aluminium hydride dropwise at −78° C. and stirred for 1 h, before quenching with solution of sodium bicarbonate. Crude product was extracted with diethyl ether (X2), the organic layers were combined and dried over sodium sulfate, filtered and concentrated in vacuo, which was purified by silica gel chromatography with hexanes and 5% Ethyl acetate/hexanes to afford the allylic alcohol 28, as a colorless oil (600 mg, 1.77 mmol, 91% yield).

### Preparation of (E)-2-methyl-3-(3-(2-((triisopropylsilyl)oxy)ethyl)bicyclo[1.1.1]pentan-1-yl)acrylaldehyde (29)

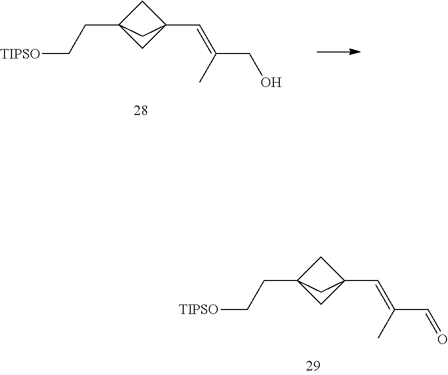

To a solution of alcohol (MW 338, 542 mg, 1.60 mmol) in 10 ml of dry dichloromethane was added Dess-Martins' periodinane (MW 424.14, 680 mg, 1.60 mmol) at ice bath temperature and the resulting reaction mixture was allowed to stir at room temperature for 2 h. Afterwards, the reaction was quenched with 2 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 20% Ethyl acetate/Hexanes to afford the aldehyde 29, as a colorless oil (460 mg, 1.37 mmol, 88% yield). The material was used in the next step without further purification.

### Preparation of (E)-triisopropyl(2-(3-(2-methylbuta-1,3-dien-1-yl)bicyclo[1.1.1]pentan-1-yl)ethoxy)silane (30)

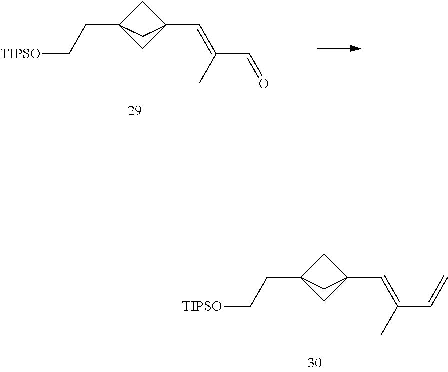

To a solution of conjugated aldehyde 29 (MW 336, 350 mg, 1.04 mmol) in 12 ml of dry diethyl ether was added 0.30 M solution of methylenetriphenylphosphorane CAS 3487-44-3 (6.0 mL, 1.8 mmol) dropwise at −78° C. The reaction mixture was allowed to stir at ice bath temperature for addition 1 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of hexanes and 5% ethyl acetate/hexanes to afford the diene 30, as a colorless oil (205 mg, 0.61 mmol, 59% yield).

### Preparation of ethyl 4-methyl-3-(3-(2-((triisopropylsilyl)oxy)ethyl)bicyclo[1.1.1]pentan-1-yl)cyclohexa-1,4-diene-1-carboxylate (31)

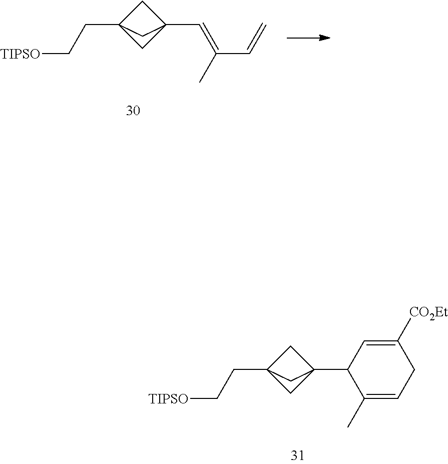

To a solution of diene 30 (MW 334, 200 mg, 0.60 mmol) and ethyl propiolate (MW 98, 150 mg, 1.5 mmol) in 4 ml of chloroform was added 1.0 M solution of diethylaluminium chloride CAS (MW 120.56, 0.6 mL, 0.6 mmol) and allow to stir at room temperature for 4 h. Afterwards, the reaction was quenched with 1 mL solution of sodium bicarbonate. Crude product was extracted with ethyl acetate (X2), the organic layers were combined and dried over sodium sulfate, filtered and concentrated in vacuo, to afford the crude 1,4-diene 31 (310 mg). The material was used in the next step without further purification.

### Preparation of ethyl 4-methyl-3-(3-(2-((triisopropylsilyl)oxy)ethyl)bicyclo[1.1.1]pentan-1-yl)benzoate (32)

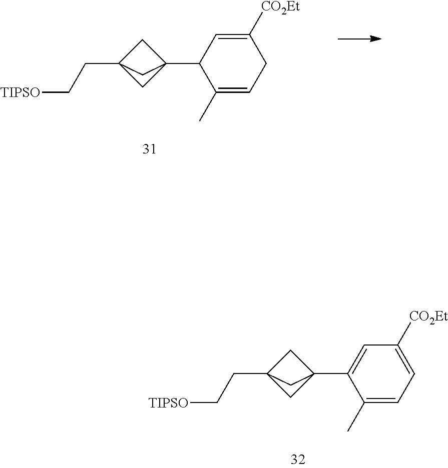

To a solution of 1,4-diene 31 (MW 432, crude 310 mg) in 4 ml of dichloromethane was added 2,3-dichloro-5,6-dicyano-1,4-benzoquinone (DDQ, MW 231, 150 mg, 0.65 mmol) at room temperature. The reaction mixture turn dark immediately and it was allowed to stir for 2 h. Afterwards, solution of sodium bicarbonate was added and the crude product was extracted with diethyl ether (X2), the organic layers were combined and dried over sodium sulfate, filtered and concentrated in vacuo. The material was purified by silica gel chromatography with elution of hexanes and 5% ethyl acetate/hexanes to afford the ethyl benzoate ester 32, as a colorless oil (130 mg, 0.30 mmol, 50% yield over 2 steps from the diene).

¹HNMR (500 MHz, CDCl₃) δ ppm 7.68 (d, J=8.0 Hz, 1H), 7.67 (s, 1H), 7.19 (t, J=8.0 Hz, 1H), 4.28 (q, J=7.0 Hz, 2H), 3.69 (q, J=6.5, 2H), 2.35 (s, 3H), 2.01 (s, 6H), 1.72 (t, J=7.0, 2H), 1.31 (t, 7.0 Hz, 6H), 1.00 (d, J=7.0 Hz, 18H).

### Preparation of ethyl 3-(3-(2-hydroxyethyl)bicyclo[1.1.1]pentan-1-yl)-4-methylbenzoate (33)

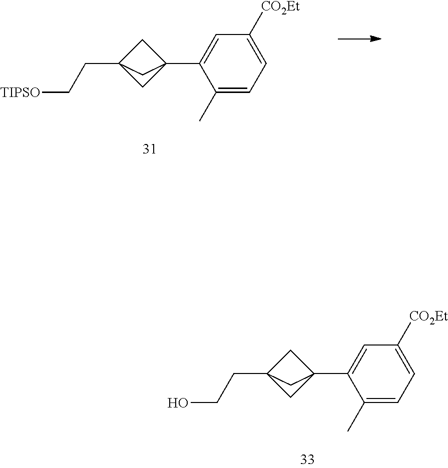

To a solution of TIPS-protected ether 31 (MW 430, 900 mg, 2.09 mmol) in 18 ml of tetrahydrofuran was added 1M solution of tetrabutylammonium fluoride (3.5 mL, 3.5 mmol) at −20° C. The reaction mixture was allowed to stir at room temperature for addition 4 h, before quenching with 2 mL solution of sodium bicarbonate. Crude product was extracted with diethyl ether (X2), the organic layers were combined and dried over sodium sulfate, filtered and concentrated in vacuo, which was purified by silica gel chromatography with elution of 10-25% Ethyl acetate/Hexanes to afford the primary alcohol 33, as a colorless oil (535 mg, 1.95 mmol, 93% yield).

### Preparation of ethyl 4-methyl-3-(3-(2-oxoethyl)bicyclo[1.1.1]pentan-1-yl)benzoate (34)

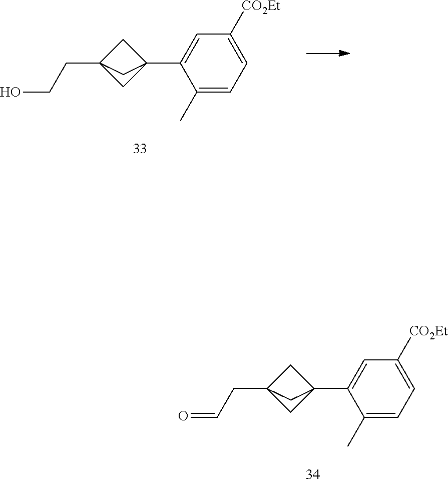 [SMILES: CCOC(=O)c1ccc(C)c(C23CC(CCO)(C2)C3)c1 ; CCOC(=O)c1ccc(C)c(C23CC(CC=O)(C2)C3)c1]

To a solution of alcohol 33 (MW 274, 300 mg, 1.09 mmol) in 20 ml of dry dichloromethane was added Dess-Martins' periodinane (MW 424.14, 525 mg, 1.24 mmol) at ice bath temperature and the resulting reaction mixture was allowed to stir at room temperature for 2 h. Afterwards, the reaction was quenched with 5 mL solution of sodium bicarbonate. Crude product was extracted with diethyl ether (X2), the organic layers were combined and dried over sodium sulfate, filtered and concentrated in vacuo, which was purified by passing through a short pad of silica gel with elution of 20% Ethyl acetate/Hexanes to afford the aldehyde 34, as a colorless oil (232 mg, 78% yield). The material was used in the next step without further purification.

### Preparation of ethyl 3-(3-(1-bromo-2-oxoethyl)bicyclo[1.1.1]pentan-1-yl)-4-methyl benzoate (35)

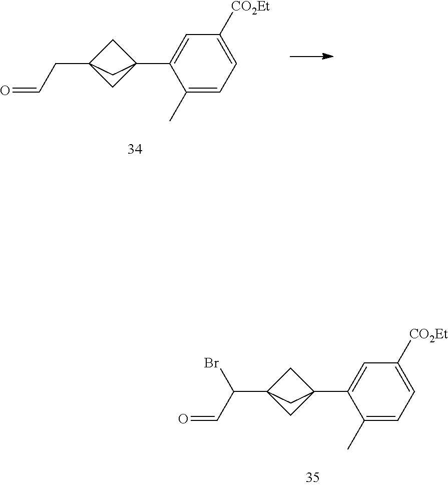 [SMILES: CCOC(=O)c1ccc(C)c(C23CC(CC=O)(C2)C3)c1 ; CCOC(=O)c1ccc(C)c(C23CC(C(Br)C=O)(C2)C3)c1]

To a solution of crude aldehyde 34 (MW 272, 232 mg, 0.85 mmol) in 6 ml of dry tetrahydrofuran was added phenyltrimethylammonium tribromide (MW 376, 336 mg, 0.90 mmol) at room temperature and it was allowed to stir at room temperature for 2 h. Afterwards, the reaction mixture was quenched with 2 mL solution of sodium bicarbonate. Crude product was extracted with diethyl ether (X2), the organic layers were combined and dried over sodium sulfate, filtered and concentrated in vacuo, to afford the crude α-bromoaldehyde 35 as an orange brownish oil (400 mg). The material was used in the next step without further purification.

### Heterocycle Formations.

The scheme 5 describes the range of synthetic coupling partners, including amides and anilines, for bromoaldehydes 15 and 35 to react with in the preparation of various heteroaryl ring systems as the target compounds of interest.

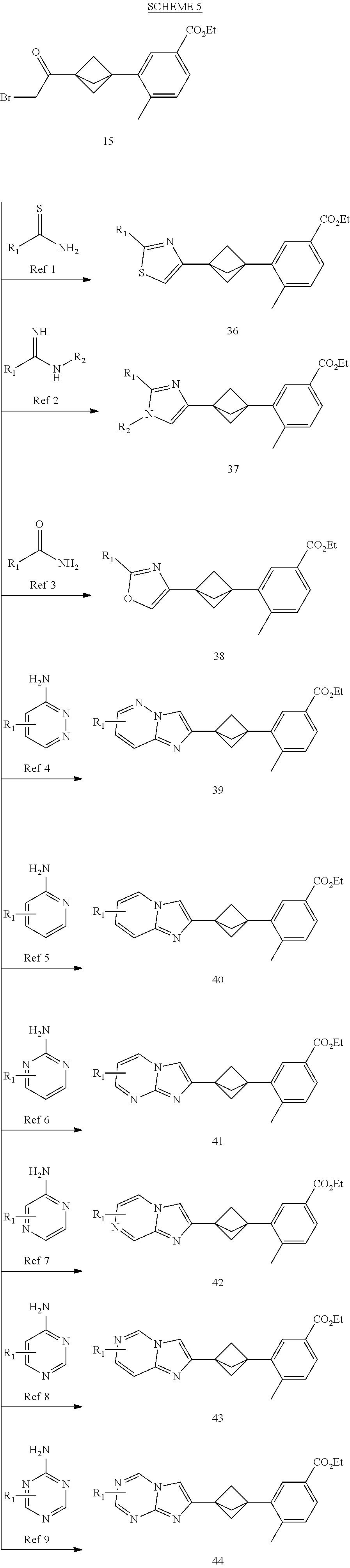

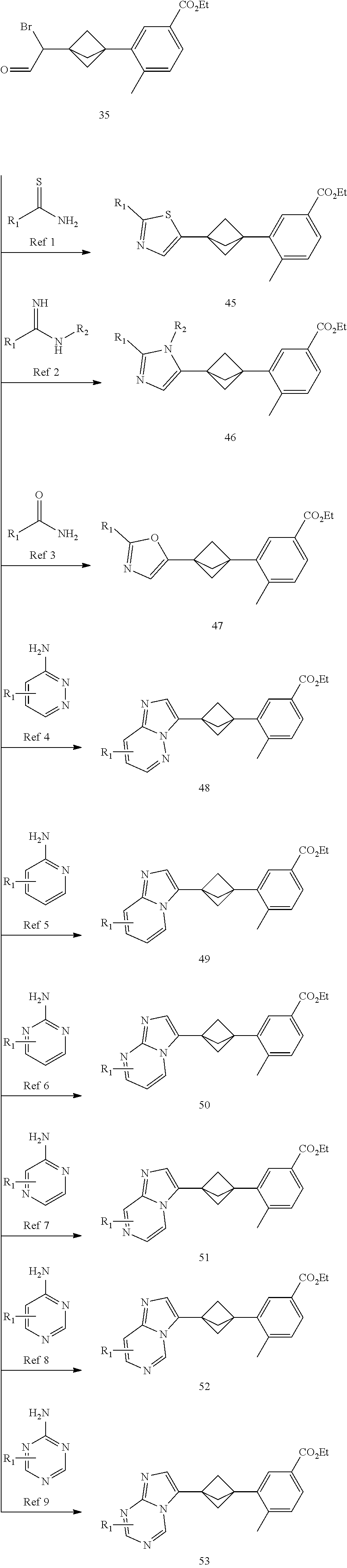

### REFERENCES FOR SCHEME 5

1) a) Facchinetti V., et al., Synthesis, 2016, 48, 437-440. An Eco-friendly, Hantzsch-Based, Solvent-Free Approach to 2-Aminothiazoles and 2-Aminoselenazoles. b) Togo H., et al., Green and Sustainable Chemistry, 2011, 1, 54-62. Preparation of α-Bromoketones and Thiazoles from Ketones with NBS and Thioamides in Ionic Liquids.
2) a) Adib, M., et. al., Synlett, 2009, 3263-3266. A One-Pot, Four-Component Synthesis of N-Substituted 2,4-Diarylimidazoles. b) Little, T. L.; et al., The Journal of Organic Chemistry, 1994, 59(24), 7299-7305. A Simple and Practical Synthesis of 2-Aminoimidazoles.
3) a) Gao, W., et al.; Organic & Biomolecular Chemistry, 11(41), 7123. Practical oxazole synthesis mediated by iodine from α-bromoketones and benzylamine derivatives; b) Peshakova L. S., 2—Chemistry of Heterocyclic Compounds 1981, 17, 741-753. Aminooxazoles and their derivatives (review)
4) Zeng F., et al., ACS Med. Chem. Lett. 2010, 1, 80-84. Synthesis and In Vitro Evaluation of Imidazo[1,2-b]-pyridazines as Ligands for β-Amyloid Plaques.
5) a) Bagdi A. K., Chemm Commum 2015, 51-1555-1575. Synthesis of imidazo[1,2-a]pyridines—a decade update; b) Chunavala K. C., Synthesis, 2011, 635-641. Thermal and Microwave-Assisted Rapid Syntheses of Substituted Imidazo[1,2-a]pyridines Under Solvent- and Catalyst-Free Conditions.
6) a) Goel, R., et al., RSC Adv, 2015, 5, 81608-81637. Synthetic approaches and functionalizations of imidazo[1,2-a]pyrimidines: an overview of the decade. b) Steenackers, H. P. L.; et al., Journal of Medicinal Chemistry, 2011, 54(2), 472-484. Structure-Activity Relationship of 4(5)-Aryl-2-amino-1H-imidazoles, N1-Substituted 2-Aminoimidazoles and Imidazo[1,2-a]pyrimidinium Salts as Inhibitors of Biofilm Formation by Salmonella Typhimurium and Pseudomonas aeruginosa
7) Goel, R., et al., Org. Biomol. Chem., 13(12), 3525-3555. Recent advances in development of imidazo[1,2-a]pyrazines: synthesis, reactivity and their biological applications. b) Bartolome-Nebreda J. M., et al., J. Med. Chem., 2014, 57, 4196. Discovery of a Potent, Selective, and Orally Active Phosphodiesterase 10A Inhibitor for the Potential Treatment of Schizophrenia.
8) a) Mazur, I. A., et al., Usp. Khim 1977, 46, 7, 1233-1249. Methods of Synthesis of Imidazopyrimidines with a Bridgehead Nitrogen Atom and Their Benzo-analogues. b) Russian Chem Review 1977, 46, 7, 634-642. b) Kifli N., et al., Bioorg. Med. Chem. 2004, 12(15), 4245-4252. Novel imidazo[1,2-c]pyrimidine base-modified nucleosides: synthesis and antiviral evaluation.
9) a) Pan Z., et al; New Journal of Chemistry, 2020, 44, 6182-6185. b) Dao, P., et al., Journal of Medicinal Chemistry, 2015, 58(1), 237-251. Design, Synthesis, and Evaluation of Novel Imidazo[1,2-a][1,3,5]triazines and their Derivatives as Focal Adhesion Kinase Inhibitors with Antitumor Activity.

While the invention has been described and illustrated with reference to certain particular embodiments thereof, those skilled in the art will appreciate that various adaptions, changes, modifications, substitutions, deletions or additions of procedures and protocols may be made without departing from the spirit and scope of the invention.

### Bromocarbonyl Cyclization Reactions

### General Procedure:

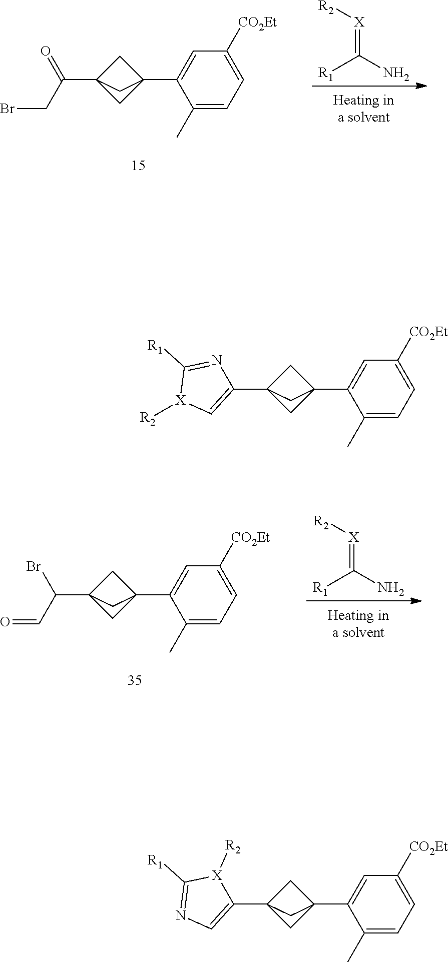

To a solution of bromoaldehyde (15 or 35) (MW 351, 1.0 mmol) in 6 ml of isopropanol is added amino containing compounds (in form of aminoheterocycles or amides) (1.5 mmol) and the resulting mixture is heated at 100° C. for 16 h. [Note: Heating in the presence of a base (i.e. trimethylamine, potassium carbonate) or under microwave conditions can speed up the reaction to completion in some cases]. Afterwards, the reaction mixture is concentrated in vacuo and the crude material can be purified by silica gel chromatography with elution of 25-50% ethyl acetate/hexanes to afford cyclized product.

### A Representative Example: Preparation of ethyl 3-(3-(imidazo(1,2-b)pyridazin-3-yl)bicyclo[1.1.1]pentan-1-yl)-4-methylbenzoate (48)

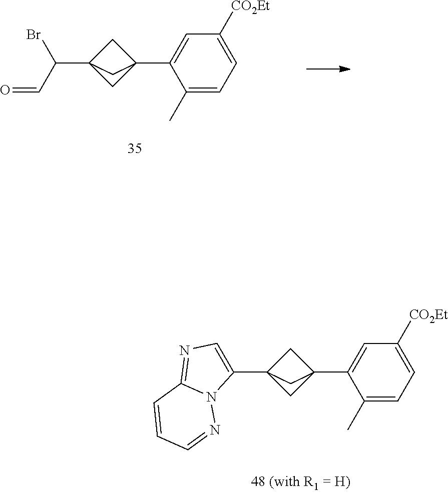 [SMILES: CCOC(=O)c1ccc(C)c(C23CC(C(Br)C=O)(C2)C3)c1 ; CCOC(=O)c1ccc(C)c(C23CC(c4cnc5cccnn45)(C2)C3)c1]

To a solution of bromoaldehyde 35 (MW 351, crude 400 mg) in 6 ml of isopropanol was added 3-aminopyridazine (MW 95, 150 mg, 1.58 mmol) and the resulting mixture was heated at 100° C. for 16 h. Afterwards, the reaction mixture was concentrated in vacuo and the crude material was purified by silica gel chromatography with elution of 25-50% ethyl acetate/hexanes to afford the imidazopyridazine 48, as a waxy solid (225 mg, 0.65 mmol, 59% yield over 2 steps from the aldehyde).

¹HNMR (500 MHz, CDCl₃) δ ppm 8.35 (s, 1H), 7.85 (d, 1H), 7.78 (s, 1H), 7.75 (d, 1H), 7.55 (s, 1H), 7.10 (d, 1H), 6.90 (d, 1H), 4.30 (q, 2H), 2.70 (s, 6H), 2.45 (s, 3H), 1.30 (t, 3H).

### Final Steps in the Synthesis of Target Compounds.

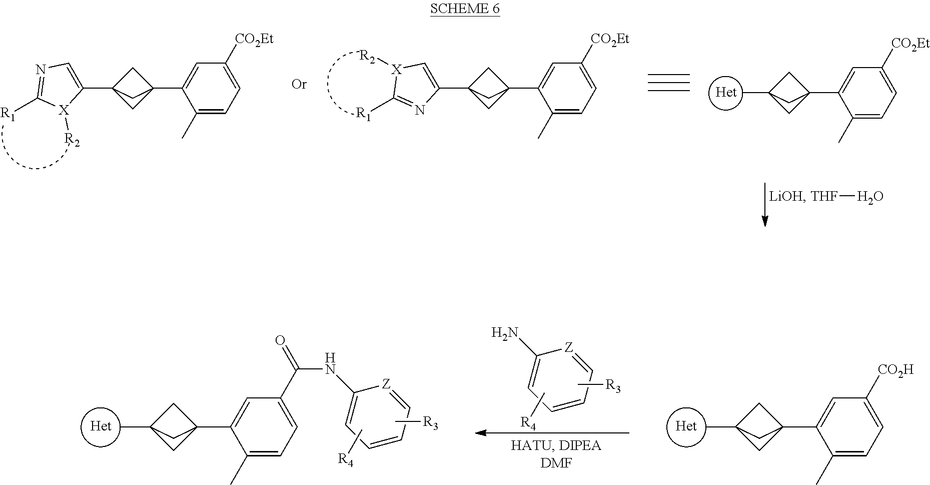

Therefore, ring Het is define as a monocyclic or a bicyclic heterocycle ring system (See scheme 6). With the heteroaromatic benzoate esters in hand, they can be easily converted to the corresponding carboxylic acid by basic hydrolysis with aqueous solution lithium hydroxide, and then subsequently coupling with an amine under a standard peptide coupling conditions (i.e HATU, DIPEA, DMF) to afford the target compounds as our novel series of inhibitors

### Step 1—Saponification of Ethyl Benzoate Esters

### General Procedure:

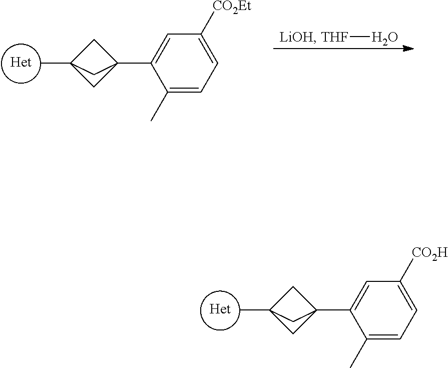

To a solution of ethyl ester (1 mmol) in 10 ml of tetrahydrofuran is added aqueous solution of lithium hydroxide monohydrate (MW 42, 113 mg, 2.68 mmol, in 4.5 ml water) and the resulting mixture is allowed to stir at room temperature for 48 h. Afterwards, the reaction mixture is neutralized with glacial acetic acid (MW 60, 0.3 ml), and the crude product was extracted with ethyl acetate (X3), the organic layers were combined and dried over sodium sulfate, filtered and concentrated in vacuo, to afford the crude acid. The material was used in the next step without further purification.

### A Representative Example: Preparation of 3-(3-(imidazo[1,2-b]pyridazin-3-yl)bicyclo[1.1.1]pentan-1-yl)-4-methylbenzoic acid (54)

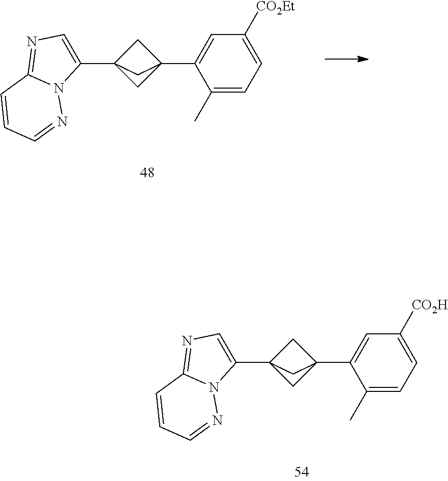 [SMILES: CCOC(=O)c1ccc(C)c(C23CC(c4cnc5cccnn45)(C2)C3)c1 ; Cc1ccc(C(=O)O)cc1C12CC(c3cnc4cccnn34)(C1)C2]

To a solution of ethyl ester (48, R₁=H)) (MW 347, 110 mg, 0.31 mmol) in 3 ml of tetrahydrofuran was added aqueous solution of lithium hydroxide monohydrate (MW 42, 35 mg, 0.83 mmol, in 1.5 ml water) and the resulting mixture was allowed to stir at room temperature for 48 h. Afterwards, the reaction mixture with neutralized was glacial acetic acid (MW 60, 0.1 ml), and the crude product was extracted with ethyl acetate (X3), the organic layers were combined and dried over sodium sulfate, filtered and concentrated in vacuo, to afford the crude acid 54, as a colorless waxy solid (105 mg). The material was used in the next step without further purification.

¹HNMR (500 MHz, CDCl₃) δ ppm 8.35 (s, 1H), 8.0 (d, 1H), 7.90 (s, 1H), 7.85 (d, 1H), 7.62 (s, 1H), 7.19 (d, 1H), 7.00 (d, 1H), 2.73 (s, 6H), 2.55 (s, 3H), 2.00 (b, 1H)

### Step 2: Carboxylic Acid—Amine Couplings to Form the Amide Functionality

### General Procedure:

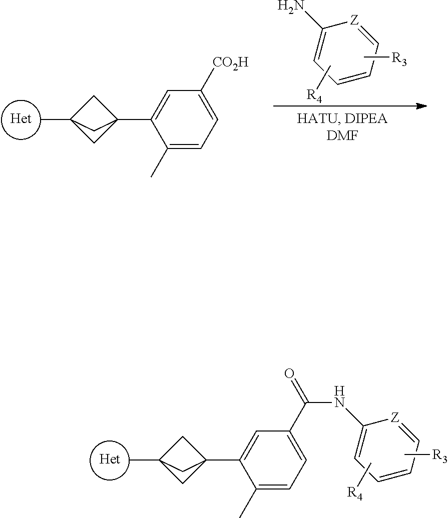

To a solution of acid (1.0 mmol), heterocyclic aniline or amide (1.0 mmol) and diisopropylethylamine (MW 129, 322 mg, 2.5 mmol) in 6 ml of N,N-dimethylformamide, was added 1-[bis(dimethylamino)methylene]-1H-1,2,3-triazolo[4,5-b]pyridinium 3-oxide hexafluorophosphate (HATU) (MW 380, 380 mg, 1.00 mmol) at room temperature and the resultant mixture was stirred for 48 h. Afterwards, 10 ml of water was added to dilute the reaction, and then mixture was extracted with dichloromethane (X3), the organic layers were combined and dried over sodium sulfate, filtered and concentrated in vacuo. The crude residue was purified silica gel chromatography by elution with 1-5% methanol/dichloromethane to afford the final target amide compound.

### A Representative Example: Preparation of 3-(3-(imidazo[1,2-b]pyridazin-3-yl)bicyclo[1.1.1]pentan-1-yl)-4-methyl-N-(4-((4-methylpiperazin-1-yl)methyl)-3-(trifluoromethyl)phenyl)benzamide (55)

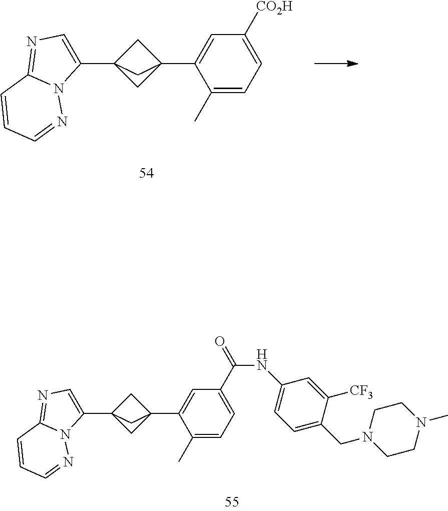 [SMILES: Cc1ccc(C(=O)O)cc1C12CC(c3cnc4cccnn34)(C1)C2 ; Cc1ccc(C(=O)Nc2ccc(CN3CCN(C)CC3)c(C(F)(F)F)c2)cc1C12CC(c3cnc4cccnn34)(C1)C2]

To a solution of benzoic acid 54 (MW 319, crude 105 mg, approx. 0.31 mmol), 4-((4-methylpiperazin-1-yl)methyl)-3-(trifluoromethyl)aniline (MW 259, 82 mg, 0.31 mmol) and diisopropylethylamine (MW 129, 95 mg, 0.73 mmol) in 2 ml of N,N-dimethylformamide, was added 1-[bis(dimethylamino)methylene]-1H-1,2,3-triazolo[4,5-b]pyridinium 3-oxide hexafluorophosphate (HATU) (MW 380, 125 mg, 0.32 mmol) at room temperature and the resultant mixture was stirred for 48 h. Afterwards, 3 ml of water was added to dilute the reaction, and then mixture was extracted with dichloromethane (X3), the organic layers were combined and dried over sodium sulfate, filtered and concentrated in vacuo. The crude residue was purified silica gel chromatography by elution with 1-5% methanol/dichloromethane to afford the final target compound 55, 3-(3-(imidazo[1,2-b]pyridazin-3-yl)bicyclo[1.1.1]pentan-1-yl)-4-methyl-N-(4-((4-methylpiperazin-1-yl)methyl)-3-(trifluoromethyl)phenyl)benzamide (80 mg, 0.14 mmol, 45% yield over 2 steps from the ethyl ester).

¹HNMR (600 MHz, CDCl₃) δ ppm ¹HNMR (500 MHz, CDCl₃) δ ppm 8.35 (s, 1H), 7.95 (m, 4H), 7.73 (d, 1H), 7.70 (s, 1H), 7.65 (d, 1H), 7.62 (d, 1H), 7.23 (d, 1H), 7.00 (d, 1H), 3.65 (s, 2H), 2.73 (s, 6H), 2.60 (m, 8H), 2.55 (s, 3H), 2.40 (s, 3H).

While the invention has been described and illustrated with reference to certain particular embodiments thereof, those skilled in the art will appreciate that various adaptions, changes, modifications, substitutions, deletions or additions of procedures and protocols may be made without departing from the spirit and scope of the invention.

### A List of Represented Compounds

Disclosed herein are examples of novel [1.1.1] bicyclo compounds of formula (I) and (II), which are inhibitors of tyrosine kinase such as Bcr-Abl. Also disclosed herein are potential uses of these compounds in the treatment of CML and ALL patients in chronic, accelerated or blast phases of the disease.

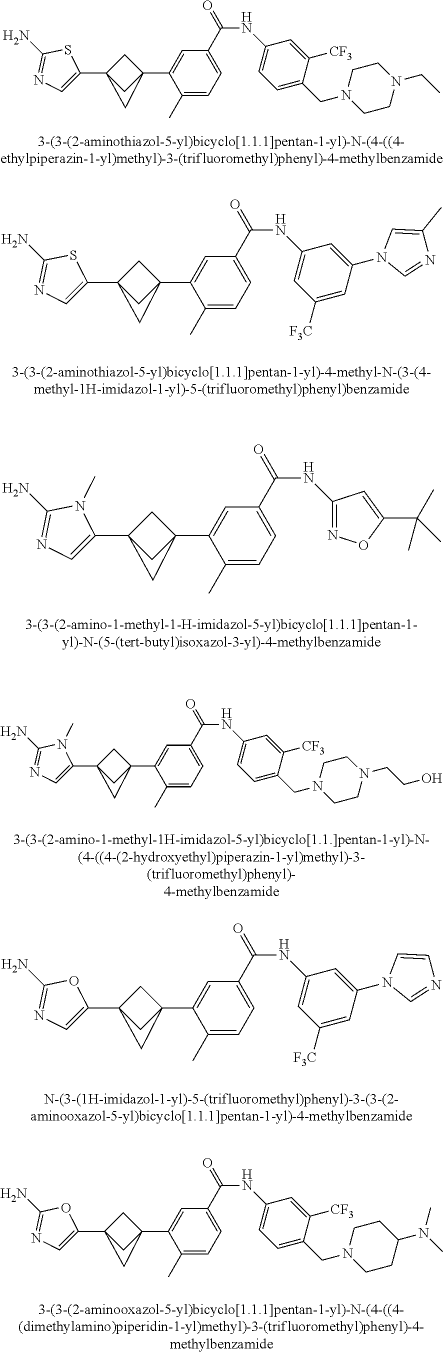 [SMILES: CCN1CCN(Cc2ccc(NC(=O)c3ccc(C)c(C45CC(c6cnc(N)s6)(C4)C5)c3)cc2C(F)(F)F)CC1 ; Cc1cn(-c2cc(NC(=O)c3ccc(C)c(C45CC(c6cnc(N)s6)(C4)C5)c3)cc(C(F)(F)F)c2)cn1 ; Cc1ccc(C(=O)Nc2cc(C(C)(C)C)on2)cc1C12CC(c3cnc(N)n3C)(C1)C2 ; Cc1ccc(C(=O)Nc2ccc(CN3CCN(CCO)CC3)c(C(F)(F)F)c2)cc1C12CC(c3cnc(N)n3C)(C1)C2 ; Cc1ccc(C(=O)Nc2cc(-n3ccnc3)cc(C(F)(F)F)c2)cc1C12CC(c3cnc(N)o3)(C1)C2 ; Cc1ccc(C(=O)Nc2ccc(CN3CCC(N(C)C)CC3)c(C(F)(F)F)c2)cc1C12CC(c3cnc(N)o3)(C1)C2]

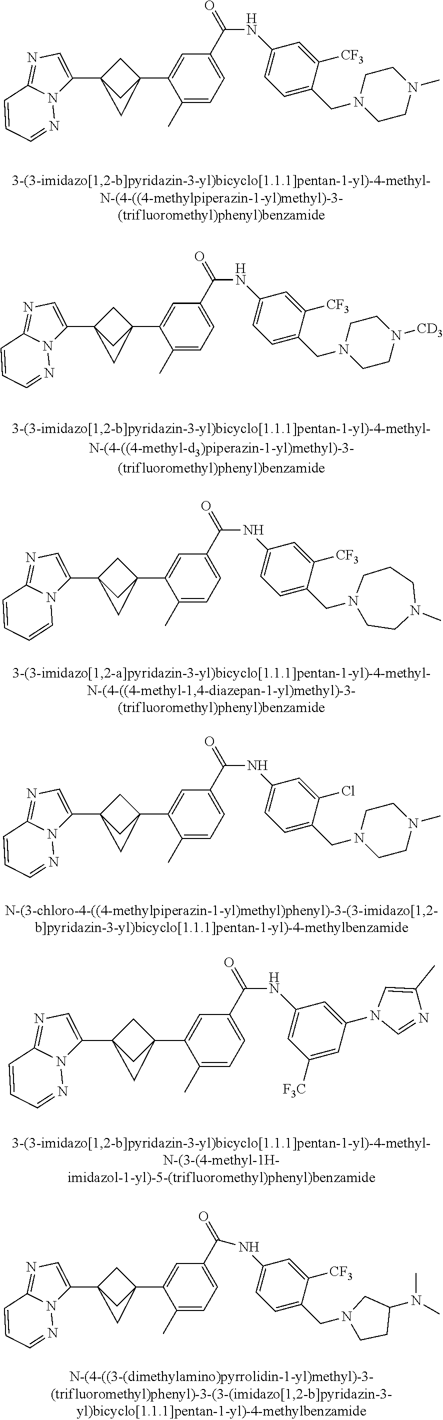 [SMILES: Cc1ccc(C(=O)Nc2ccc(CN3CCN(C)CC3)c(C(F)(F)F)c2)cc1C12CC(c3cnc4cccnn34)(C1)C2 ; [2H]C([2H])([2H])N1CCN(Cc2ccc(NC(=O)c3ccc(C)c(C45CC(c6cnc7cccnn67)(C4)C5)c3)cc2C(F)(F)F)CC1 ; Cc1ccc(C(=O)Nc2ccc(CN3CCCN(C)CC3)c(C(F)(F)F)c2)cc1C12CC(c3cnc4ccccn34)(C1)C2 ; Cc1ccc(C(=O)Nc2ccc(CN3CCN(C)CC3)c(Cl)c2)cc1C12CC(c3cnc4cccnn34)(C1)C2 ; Cc1cn(-c2cc(NC(=O)c3ccc(C)c(C45CC(c6cnc7cccnn67)(C4)C5)c3)cc(C(F)(F)F)c2)cn1 ; Cc1ccc(C(=O)Nc2ccc(CN3CCC(N(C)C)C3)c(C(F)(F)F)c2)cc1C12CC(c3cnc4cccnn34)(C1)C2]

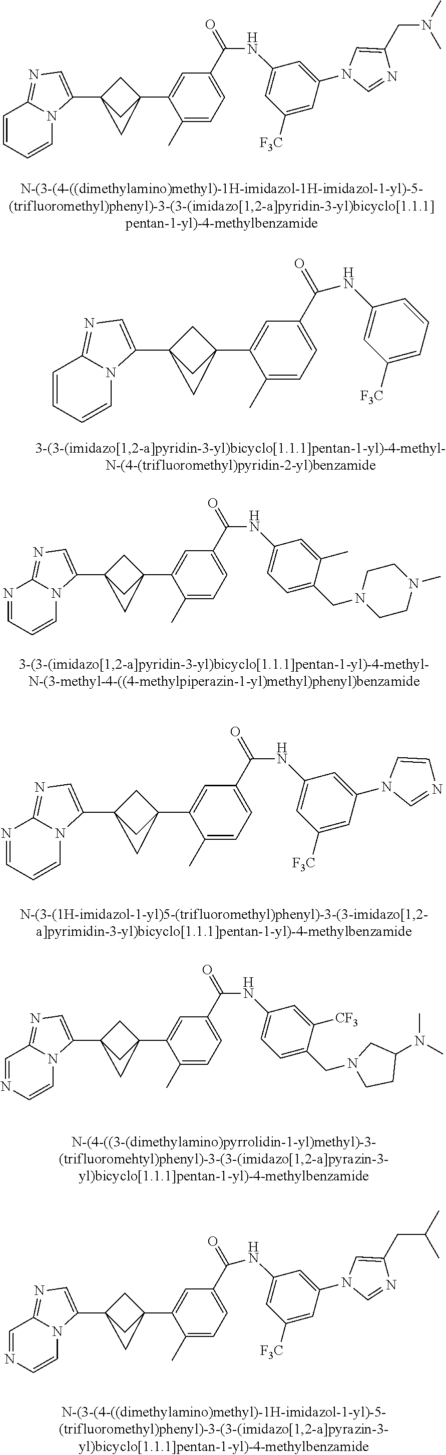 [SMILES: Cc1ccc(C(=O)Nc2cc(-n3cnc(CN(C)C)c3)cc(C(F)(F)F)c2)cc1C12CC(c3cnc4ccccn34)(C1)C2 ; Cc1ccc(C(=O)Nc2cccc(C(F)(F)F)c2)cc1C12CC(c3cnc4ccccn34)(C1)C2 ; Cc1cc(NC(=O)c2ccc(C)c(C34CC(c5cnc6ncccn56)(C3)C4)c2)ccc1CN1CCN(C)CC1 ; Cc1ccc(C(=O)Nc2cc(-n3ccnc3)cc(C(F)(F)F)c2)cc1C12CC(c3cnc4ncccn34)(C1)C2 ; Cc1ccc(C(=O)Nc2ccc(CN3CCC(N(C)C)C3)c(C(F)(F)F)c2)cc1C12CC(c3cnc4cnccn34)(C1)C2 ; Cc1ccc(C(=O)Nc2cc(-n3cnc(CC(C)C)c3)cc(C(F)(F)F)c2)cc1C12CC(c3cnc4cnccn34)(C1)C2]

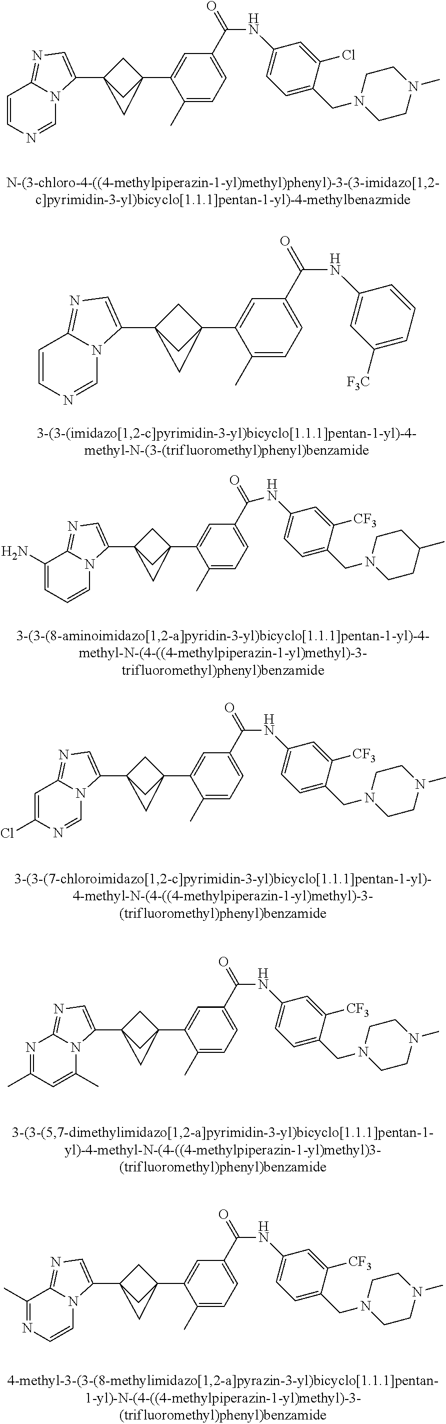

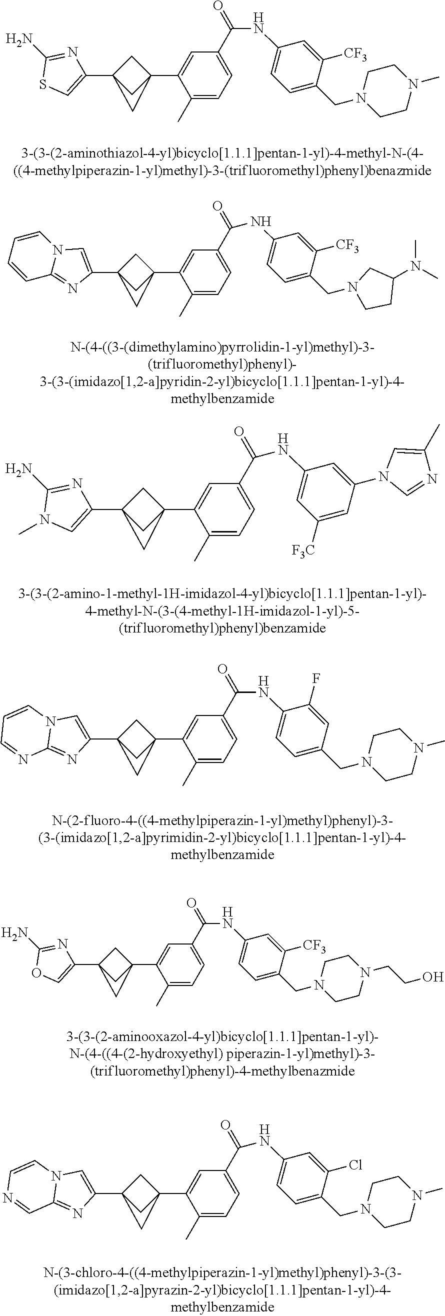

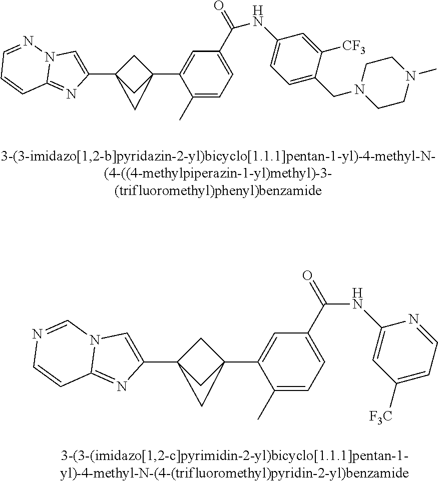 [SMILES: Cc1ccc(C(=O)Nc2ccc(CN3CCN(C)CC3)c(C(F)(F)F)c2)cc1C12CC(c3cn4ncccc4n3)(C1)C2 ; Cc1ccc(C(=O)Nc2cc(C(F)(F)F)ccn2)cc1C12CC(c3cn4cnccc4n3)(C1)C2]

However the series of inhibitors are not limited to the structures listed above. While the invention has been described and illustrated with reference to certain particular embodiments thereof, those skilled in the art will appreciate that various adaptions, changes, modifications, substitutions, deletions or additions of procedures and protocols may be made without departing from the spirit and scope of the invention.

### Biological Assays

### Assay was conducted by BPS Biosciences, 6405 Mira Mesa Blvd. Suite 100. San Diego, Calif. 92121. United States

### Assay Conditions and Analysis

### Enzymes and Substrates

| | Catalog # | Enzyme Used | |
|---|---|---|---|
| Assay | (Lot #) | (ng)/Reaction | Substrate |
| Abl | 40411 (#141106) | 5 | 0.1 mg/ml Abltide/ |
| | | | 10 μM ATP |
| Abl(T315I) | 40415 (#80828) | 10 | 0.1 mg/ml Abltide/ |
| | | | 10 μM ATP |

The assay was performed using Kinase-Glo Plus luminescence kinase assay kit (Promega). It measures kinase activity by quantitating the amount of ATP remaining in solution following a kinase reaction. The luminescent signal from the assay is correlated with the amount of ATP present and is inversely correlated with the amount of kinase activity.
The testing compounds were diluted in 10% DMSO and 5 μl of the dilution was added to a 50 μl reaction so that the final concentration of DMSO is 1% in all of reactions. All of the enzymatic reactions were conducted at 30° C. for 45 minutes. The 50 μl reaction mixture contains 40 mM Tris, pH 7.4, 10 mM MgCl₂, 0.1 mg/ml BSA, 1 mM DTT, 10 μM ATP, Kinase substrate and the enzyme. After the enzymatic reaction, 50 μl of Kinase-Glo Plus Luminescence kinase assay solution (Promega) was added to each reaction and incubated on the plate for 15 minutes, at room temperature. Luminescence signal was measured using a BioTek Synergy 2 microplate reader.

### Data Analysis

Kinase activity assays were performed in duplicate at each concentration. The luminescence data were analyzed using the computer software, Graphpad Prism. The difference between luminescence intensities in the absence of Kinase (Luₜ) and in the presence of Kinase (Lu_{c}) was defined as 100% activity (Luₜ−Lu_{c}). Using luminescence signal (Lu) in the presence of the compound, % activity was calculated as:
% activity={(Luₜ−Lu)/(Luₜ−Lu_{c})}×100%, where Lu=the luminescence intensity in the presence of the compound. The values of % activity versus a series of compound concentrations were then plotted using non-linear regression analysis of Sigmoidal dose-response curve generated with the equation Y=B+(T−B)/1+10 ((LogEC50−X)×Hill Slope), where Y=percent activity, B=minimum percent activity, T=maximum percent activity, X=logarithm of compound and Hill Slope=slope factor or Hill coefficient. The IC 50 value was determined by the concentration causing a half-maximal percent activity.

### Biological Activity of Compound 55

Data for the inhibition effect of the compound 55 on kinase activity of ABL(WT) and ABL(T315I).

% Inhibition of Compound 55

| ASSAY | @10 nM | @100 nM |
|---|---|---|
| ABL (WT) | >10% | >25% |
| ABL (T3I5I) | >10% | >25% |

IC 50 of the compound 55 against ABL (WT) and ABL (T315I)

| ASSAY | IC-50 of Compound 55 |
|---|---|
| ABL (WT) | <500 nM |
| ABL (T3I5I) | <500 nM |

1. A compound of the Formula I or a tautomer or an individual enantiomeric isomer thereof in which: 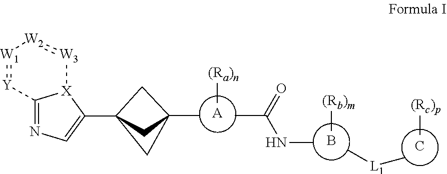
X is selected from NRₓ, O or S;
Rₓ is selected from H, C1-4 alkyl, C1-4 haloalkyl or a covalent bond, if X and Y together with the atoms which X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
Ring A represents a 5- or 6 membered aryl or heteroaryl ring system, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a 5- or 6 membered aryl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);
L₁ is selected from —(CH₂)_{q}— (where q=1-2), O or a covalent bond;
Ring C represents a 5- or 6 membered aryl, heteroaryl, cycloalkyl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

2. A compound of the Formula II or a tautomer or an individual enantiomeric isomer thereof in which: 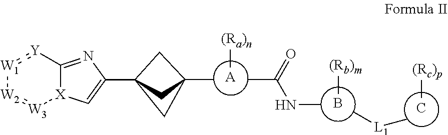
X is selected from NRₓ, O or S;
Rₓ is selected from H, C1-4 alkyl, C1-4 haloalkyl or a covalent bond, if X and Y together with the atoms which X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
Ring A represents a 5- or 6 membered aryl or heteroaryl ring system, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a 5- or 6 membered aryl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);
L₁ is selected from —(CH₂)_{q}— (where q=1-2), O or a covalent bond;
Ring C represents a 5- or 6 membered aryl, heteroaryl, cycloalkyl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

3. A compound of claim 1 wherein:
Ring A represents a phenyl, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a phenyl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);
Ring C represents a 5- or 6 membered heteroaryl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

4. A compound of claim 2 wherein:
Ring A represents a phenyl, and is substituted with Rₐ group, where n=0-3;
Rₐ, at each occurrence, is independently selected from a group consisting of halogen, CH₃, CD₃, CF₃, C2-4 alkyl (branched or unbranched) or C1-4 haloalkyl (branched or unbranched);
Ring B represents a phenyl or heteroaryl ring system, and is substituted with R_{b} groups, where m=0-3;
R_{b}, at each occurrence, is independently selected from a group consisting of halogen, CH₃, CD₃, CF₃, C2-4 alkyl (branched or unbranched) or haloalkyl C1-4 (branched or unbranched);
Ring C represents a 5- or 6 membered heteroaryl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

5. A compound of claim 1 selected from Formula III: 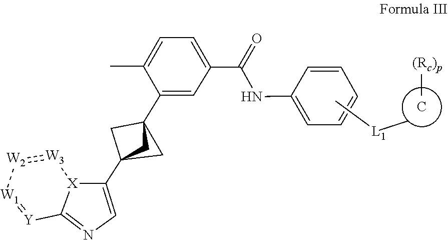
X is selected from NRₓ, O or S;
Rₓ is selected from H, C1-4 alkyl, C1-4 haloalkyl or a covalent bond, if X and Y together with the atoms which X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
L₁ is selected from —(CH₂)_{q}— (where q=1-2), O or a covalent bond;
Ring C represents a 5- or 6 membered aryl, heteroaryl, cycloalkyl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

6. A compound of claim 2 selected from Formula IV: 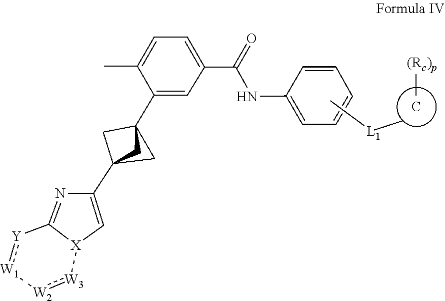
X is selected from NRₓ, O or S;
Rₓ is selected from H, C1-4 alkyl, C1-4 haloalkyl or a covalent bond, if X and Y together with the atoms which X and Y are attached form a 5,6-membered fused heteroaryl system;
Y is selected from H, C1-6 alkyl, C1-6 haloalkyl, NR₁R₂, NR₁COR₂, NR₁CONR₁R₂, NO₂, COR₁ or CONR₁R₂. However, if X and Y together with the atoms which X and Y are attached form a 5,6-membered fused heteroaryl system, then Y is CR_{w} or N;
If X and Y form a 5,6-membered fused heteroaryl system, then W₁, W₂ and W₃ are independently selected from CR_{w} or N;
R_{w}, at each occurrence, is independently selected from H, D, halogen, C1-4 alkyl (branched or unbranched), C1-4 haloalkyl (branched or unbranched), OR₁, NO₂, NR₁R₂, NR₁COR₂, NR₁CONR₁R₂, COR₁ or CONR₁R₂;
R₁ and R₂, at each occurrence, are independently selected from H, C1-6 alkyl (branched or unbranched), C1-6 haloalkyl, aryl or heteroaryl;
L₁ is selected from —(CH₂)_{q}— (where q=1-2), O or a covalent bond Ring C represents a 5- or 6 membered aryl, heteroaryl, cycloalkyl or heterocycloalkyl and is substituted with R_{c} groups, where p=0-3;
R_{c}, at each occurrence, is independently selected from a group consisting of halogen, CH₃, CD₃, CF₃, C2-4 alkyl (branched or unbranched) or haloalkyl C1-4 (branched or unbranched).

7. A compound of claim 1 selected from: 3-(3-(2-aminothiazol-5-yl)bicyclo[1.1.1]pentan-1-yl)-N-(4-((4-ethylpiperazin-1-yl)methyl)-3-(trifluoromethyl)phenyl)-4-methylbenzamide, 3-(3-(2-amino-1-methyl-1H-imidazol-5-yl)bicyclo[1.1.1]pentan-1-yl)-N-(5-(tert-butyl)isoxazol-3-yl)-4-methylbenzamide, N-(3-(1H-imidazol-1-yl)-5-(trifluoromethyl)phenyl)-3-(3-(2-aminooxazol-5-yl)bicyclo[1.1.1]pentan-1-yl)-4-methylbenzamide, 3-(3-(imidazo[1,2-b]pyridazin-3-yl)bicyclo[1.1.1]pentan-1-yl)-4-methyl-N-(4-((4-methylpiperazin-1-yl)methyl)-3-(trifluoromethyl)phenyl)benzamide, 3-(3-(imidazo[1,2-a]pyridin-3-yl)bicyclo[1.1.1]pentan-1-yl)-4-methyl-N-(4-((4-methyl-1,4-diazepan-1-yl)methyl)-3-(trifluoromethyl)phenyl)benzamide, 3-(3-(imidazo[1,2-b]pyridazin-3-yl)bicyclo[1.1.1]pentan-1-yl)-4-methyl-N-(3-(4-methyl-1H-imidazol-1-yl)-5-(trifluoromethyl)phenyl)benzamide.

8. A compound of claim 2 selected from: 3-(3-(2-aminothiazol-5-yl)bicyclo[1.1.1]pentan-1-yl)-4-methyl-N-(3-(4-methyl-1H-imidazol-1-yl)-5-(trifluoromethyl)phenyl)benzamide, 3-(3-(2-amino-1-methyl-1H-imidazol-5-yl)bicyclo[1.1.1]pentan-1-yl)-N-(4-((4-(2-hydroxyethyl)piperazin-1-yl)methyl)-3-(trifluoromethyl)phenyl)-4-methylbenzamide, 3-(3-(2-aminooxazol-5-yl)bicyclo[1.1.1]pentan-1-yl)-N-(4-((4-(dimethylamino)piperidin-1-yl)methyl)-3-(trifluoromethyl)phenyl)-4-methylbenzamide, 3-(3-(imidazo[1,2-b]pyridazin-3-yl)bicyclo[1.1.1]pentan-1-yl)-4-methyl-N-(4-((4-(methyl-d3)piperazin-1-yl)methyl)-3-(trifluoromethyl)phenyl)benzamide, N-(3-chloro-4-((4-methylpiperazin-1-yl)methyl)phenyl)-3-(3-(imidazo[1,2-b]pyridazin-3-yl)bicyclo[1.1.1]pentan-1-yl)-4-methylbenzamide, N-(4-((3-(dimethylamino)pyrrolidin-1-yl)methyl)-3-(trifluoromethyl)phenyl)-3-(3-(imidazo[1,2-b]pyridazin-3-yl)bicyclo[1.1.1]pentan-1-yl)-4-methylbenzamide.

9. A compound of claim 5 wherein the structure is 3-(3-(imidazo[1,2-b]pyridazin-3-yl)bicyclo[1.1.1]pentan-1-yl)-4-methyl-N-(4-((4-methylpiperazin-1-yl)methyl)-3-(trifluoromethyl)phenyl)benzamide (Shown as Compound 55).

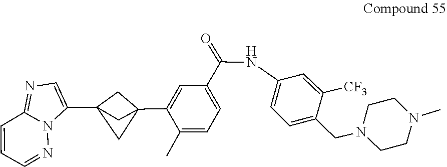 [SMILES: Cc1ccc(C(=O)Nc2ccc(CN3CCN(C)CC3)c(C(F)(F)F)c2)cc1C12CC(c3cnc4cccnn34)(C1)C2]

10. The pharmaceutical composition of claim 1, and a pharmaceutically acceptable prodrug thereof, a pharmaceutically active metabolite thereof, a pharmaceutically acceptable salt thereof and a pharmaceutical formulation thereof.

11. The pharmaceutical composition of claim 2, and a pharmaceutically acceptable prodrug thereof, a pharmaceutically active metabolite thereof, a pharmaceutically acceptable salt thereof and a pharmaceutical formulation thereof.

12. The pharmaceutical composition of claim 1, and a pharmaceutically acceptable carrier, diluent or vehicle.

13. The pharmaceutical composition of claim 2, and a pharmaceutically acceptable carrier, diluent or vehicle.

14. The pharmaceutical composition of claim 1, wherein said composition is for the treatment of a disease regulated by a protein kinase.

15. The pharmaceutical composition of claim 2, wherein said composition is for the treatment of a disease regulated by a protein kinase.

16. A method for regulating the tyrosine kinase signaling transduction comprising administration to a mammalian subject a therapeutically effective amount of a compound of claim 1.

17. A method for regulating the tyrosine kinase signaling transduction comprising administration to a mammalian subject a therapeutically effective amount of a compound of claim 2

18. A method for treating or preventing a Bcr-Abl, c-Kit or PDGFR mediated disorder or a mutant protein thereof, said method comprises administering to a mammalian subject a therapeutically effective amount of a compound of claim 1.

19. A method for treating or preventing a Bcr-Abl, c-Kit or PDGFR mediated disorder or a mutant protein thereof, said method comprises administering to a mammalian subject a therapeutically effective amount of a compound of claim 2.

20. The method or use of claim 1, wherein the leukemia is chronic myelogenous leukemia, acute lymphoblastic leukemia, acute myelogenous leukemia, chronic lymphoblastic leukemia, or imatinib-resistant chronic myelogenous leukemia.
